(19) United States
(12) Patent Application Publication
(10) Pub. No.: US 2024/0107988 A1
(43) Pub. Date: Apr. 4, 2024

(54) DOWN-REGULATION OF ENDOGENOUS GENES

(71) Applicants: UNIVERSITAT AUTÒNOMA DE BARCELONA, Cerdanyola del Vallès (ES); HELMHOLTZ ZENTRUM MÜNCHEN DEUTSCHES FORSCHUNGSZENTRUM FÜR GESUNDHEIT UND UMWELT (GMBH), Neuherberg (DE)

(72) Inventors: Maria Fátima BOSCH TUBERT, Cerdanyola del Valles (ES); Verónica JIMENEZ CENZANO, Sabadell (ES); Miquel GARCIA MARTINEZ, Terrassa (ES); Estefanía CASANA LORENTE, Sabadell (ES); Martin Matthias HRABE DE ANGELIS, Starnberg (DE); Gerhard Kurt Herbert PRZEMECK, München (DE); Anna-Lena AMEND, München (DE)

(73) Assignees: UNIVERSITAT AUTÒNOMA DE BARCELONA, Cerdanyola del Vallès (ES); HELMHOLTZ ZENTRUM MÜNCHEN DEUTSCHES FORSCHUNGSZENTRUM FÜR GESUNDHEIT UND UMWELT (GMBH), Neuherberg (DE)

(21) Appl. No.: 18/262,018

(22) PCT Filed: Jan. 27, 2022
(86) PCT No.: PCT/EP2022/051917

§ 371 Date: Jul. 19, 2023

(30) Foreign Application Priority Data
Jan. 30, 2021 (EP) 21382080.6

(51) Int. Cl. A01K 67/0278; C12N 9/22; C12N 15/11; C12N 15/90

(52) U.S. Cl. CPC A01K 67/0278; C12N 9/22; C12N 15/11; C12N 15/907; A01K 2217/054; A01K 2217/07; A01K 2227/103; A01K 2267/035; C12N 2310/20

(57) ABSTRACT

Described herein is a genetically modified plant or non-human animal having reduced expression of an endogenous target gene.

### FIELD

Aspects and embodiments decribed herein relate to the field of biotechnology, particularly genetically modified plants and non-human animals.

### BACKGROUND

Many plant and animal phenotypes, including traits, diseases and disorders, are largely determined by a single or multiple genes and may be denoted as genetic traits, diseases and disorders. In case the phenotype is attributable to a single gene it is often referred to as a monogenic trait, disease or disorder. In many cases, mutations in genes are partial or complete loss-of-function mutations, meaning that the resulting gene product has less or no function. To study genetic traits, diseases and disorders, global and unconditional knockout or knockdown approaches such as embryonic stem (ES)-cell based gene targeting, gene editing (by employing e.g. ZFNs, TALENs, or CRISPR-Cas), RNA interference (RNAi) and N-ethyl-N-nitrosourea (ENU) mutagenesis have been used. However, such approaches might be associated with several disadvantages such as embryonic lethality and do not address species-specific differences in gene expression. “Gao et al. Transgenic RNA interference in mice. Physiology 2007; 22(3):161-166” described an RNAi approach using transgenic mice expressing small hairpin RNA (shRNA). The difficulty of achieving tissue specificity and temporal control of silencing of endogenous genes by this technology as well as the toxicity issues raised by the overexpression of artificial shRNA sequences make that RNA interference-based approaches have not been broadly adopted.

Moreover, global knockout or knockdown animals often display multiple undesired side effects, which are not observed for the actual (monogenic) traits, diseases and disorders.

Some methods have been developed for the generation of tissue-specific downregulation or knock-out of genes, such as the Cre/loxP, Flp/FRT or the Dre/rox systems. However, these existing methods have the drawback that they require the insertion of exogenous sequences (recombination sites and/or selection markers), which can unintentionally deregulate transcription and splicing. Upon recombination, usually one of the recombinase-specific sites is maintained in the edited genome. Moreover, if the selection marker gene is not properly removed, its maintenance in the genetically modified genome may lead to potential off-target effects such as unwanted splicing effects or hypomorphic alleles not only in the target tissue but in the rest of tissues in which the gene of interest is endogenously expressed.These methods are also time consuming since additional crossing steps are mandatory.

In case of dominant negative allelles, specific overexpression of mutated genes in an organism has been used as a model. However, while expression of such dominant-negative variants may recapitulate the trait, disorder or disease, such models do not allow testing therapeutic or non-therapeutic interventions such as protein replacement therapy or gene therapy, because the dominant negative variant would sequester the wild-type form.

Examples of a genetic, more specifically a monogenic, disease or disorder are diseases known as maturity-onset diabetes of the young (MODY), which comprises a heterogeneous group of monogenic disorders characterized by beta-cell dysfunction (impaired insulin secretion) with minimal or no defects in insulin action. MODYs are a rare cause of diabetes (1-2% of all cases of diabetes) (Fajans, S. S. et al. (2011). Diabetes Care, 34, 1878-84), with onset of hyperglycemia at an early age (generally before 25 years) (American Diabetes Association (2014). Diabetes Care, 37 Suppl 1, S81-90). MODY3 is the most common cause of MODY and is caused by mutations in the gene encoding for the transcription factor hepatocyte nuclear factor (HNF) 1A (Anik. A (2015). J. Pediatr. Endocrinol. Metab. 28, 251-63). MODY3 patients are typically normoglycemic in childhood, but mutations in the HNF1A gene cause progressive pancreatic beta-cell dysfunction that results in hyperglycemia, which is usually diagnosed between the second and fifth decades of life (Thanabalasingham, G. et al. (2011). BMJ, 343, d6044). Consequently, MODY3 patients are at risk of development of the full spectrum of microvascular and macrovascular complications associated with diabetes (Anik. A (2015). J. Pediatr. Endocrinol. Metab. 28, 251-63, Thanabalasingham, G. et al. (2011). BMJ, 343, d6044). MODY1 is caused by mutations in the gene encoding for the transcription factor hepatic nuclear factor (HNF) 4A (Yamagata K. (1996). Nature 384, 458-460). HNF4A, similar to HNF1A, is also a transcription factor and was shown to be essential for beta-cell function and maintaining glucose homeostasis. It is involved in the regulation of genes involved in insulin secretion such as glucokinase (GCK), SLC2A2 (encoding for GLUT2) or also HNF1A. MODY1 patients often show transient neonatal hypoglycemia that later evolves to decreased insulin secretion and diabetes (Pearson E. R. et al. (2007). Macrosomia and hyperinsulinaemic hypogylcaemia in patients with heterozygous mutations in the HNF4 gene. PLoS Med. 4, e118). Due to the early onset of hyperglycemia usually before 25 years of age, MODY1 patients are equally at risk for diabetes-associated complications as MODY3 patients.

To test the therapeutic efficacy of suitable therapies such as protein replacement therapy and gene therapy approaches, MODY mouse models that reproduce the phenotype observed in patients are required. In the case of MODY3, there do exist two different global HNF1A knockout mouse models. Although these animals display a diabetic phenotype, they also show multiple organ manifestations that are not observed in MODY3 patients. In contrast, the beta-cell-specific overexpression of dominant negative mutants of HNF1A in two different lines of transgenic mice closely recapitulates the beta-cell dysfunction and diabetes observed in MODY3, without an extra-pancreatic phenotype. However, these lines cannot be used to assess the therapeutic potential of HNF1A overexpression or replacement therapies for MODY3 because in the engineered beta-cells the dominant negative mutants would sequester the wild-type form of the HNF1A protein. Moreover, in dominant negative models the mutant HNF1A protein may sequester other β-cell proteins, affecting the observed phenotype. Thus, MODY3 mouse models that exhibit a similar patient's phenotype and permit the evaluation of all feasible future therapies are required. In the case of MODY1, the existing mouse models are either homozygous lethal or show no MODY-relevant phenotype at all when heterozygous.

In view of the above, there is still a need for new types of plant and animal models that closely recapitulate monogenic traits, disorders or diseases, and permit the evaluation of all feasible therapeutic and non-therapeutic interventions.

### SUMMARY

In an aspect, the invention relates to a genetically modified plant or non-human animal having reduced expression of an endogenous target gene, wherein the genome of the plant or non-human animal is modified by ectopic integration of at least one copy of a target sequence of a microRNA. In some embodiments, the genetically modified plant or non-human animal is such that the ectopic integration site is located:
in the 5′ UTR of the target gene or in the region between the 5′ UTR and the first exon of the target gene; or
in the 3′ UTR of the target gene or in the region between the last exon and the 3′ UTR of the target gene.

In some embodiments, the ectopic integration site is located in the 3′ UTR of the target gene or in the region between the last exon and the 3′ UTR of the target gene. In some embodiments, the genetically modified plant or non-human animal is such that the genome comprises one, two, three, four, five, six, seven, eight or more copies, preferably two, three or four copies, more preferably two copies of the target sequence of a microRNA. In some embodiments, the genetically modified plant or non-human animal is such that the reduced expression is specifically in one or more target cells, tissues and/or organs of an organism, and wherein the target sequence is of a microRNA expressed specifically in said one or more target cells, tissues and/or organs. In some embodiments, the reduced expression is specifically in one or more target cells, tissues and/or organs of an organism, and the target sequence is of a microRNA expressed specifically in said one or more target cells, tissues and/or organs. In some embodiments, a genetically modified plant or non-human animal is a non-human animal, preferably a mammal such as a rodent, more preferably a rat or a mouse, most preferably a mouse. In some embodiments, the genetically modified plant or non-human animal is such that the reduced expression is specifically in the pancreas and the target sequence is of a microRNA expressed specifically in the pancreas, preferably wherein the microRNA is selected from the group consisting of: mir-200a, mir-96, mir-1839-1, mir-34a, mir-7b, mir-7-1, mir-7-2, mir-184, mir-375, mir-219a-1, mir-574, mir-802, mir-152 and mir-148a, more preferably wherein the microRNA is mir-375. In some embodiments, the genetically modified plant or non-human animal is such that the target gene as described herein is selected from the group consisting of: HNF4A, GCK, HNF1A, PDX1, HNF1B, NEUROD1, KLF11, CEL, PAX4, INS, BLK, ABCC8, APPL1 and KCNJ11, preferably the target gene is HNF1A or HNF4A. In some embodiments, the genetically modified plant or non-human animal is a plant or non-human animal disease model, preferably a model for a disease associated with or caused by mutations in the target gene, more preferably wherein the disease is a monogenic disease. In some embodiments, the disease is maturity onset diabetes of the young type 3 (MODY3) and the target gene is HNF1A, or the disease is maturity onset diabetes of the young type 1 (MODY1) and the target gene is HNF4A.

In another aspect the invention relates to a method for obtaining a genetically modified plant or non-human animal of the invention, comprising:
(a) providing a cell of said plant or non-human animal;
(b) genetically modifying the cell by ectopic integration of at least one copy of a target sequence of a microRNA in the genome of the cell;
(c) generating an embryo from the cell; and
(d) growing said embryo to form a genetically modified plant or non-human animal.

In some embodiments, the at least one copy of a target sequence of a microRNA is introduced by homology-directed repair (HDR), preferably CRISPR-mediated HDR. In some embodiments, the method for obtaining a genetically modified plant or non-human animal of the invention further comprises the step of back-crossing the genetically modified plant or non-human animal with non-genetically modified wildtype plant or non-human animal.

In another aspect the invention relates to a method for identifying and/or evaluating therapeutic efficacy of a candidate agent for treating, inhibiting or preventing a disease, comprising administering the candidate agent to a plant or non-human animal of the invention which is a plant or non-human animal disease model, more preferably a model for a disease associated with or caused by mutations in the target gene, or a cell, tissue or organ derived thereof.

In another aspect the invention relates to a method for identifying and/or evaluating therapeutic efficacy of a candidate agent for treating, inhibiting or preventing MODY3 or MODY1, comprising administering the agent to a plant or non-human animal of the invention which is a plant or non-human animal disease model, more preferably a model for a disease associated with or caused by mutations in the target gene, wherein the disease is maturity onset diabetes of the young type 3 (MODY3) and the target gene is HNF1A, or wherein the disease is maturity onset diabetes of the young type 1 (MODY1) and the target gene is HNF4A, or a cell, tissue or organ derived thereof.

### DESCRIPTION

The present inventors have developed an efficient and highly specific method to obtain reduced expression of an endogenous target gene in any plant or animal species, preferably in specific cells, tissues, and/or organs. The method is based on knocking in one or more target sites for a microRNA near the target gene. As a consequence, the target gene transcript will comprise one or more microRNA target sites that will prevent translation in any cell, tissue or organ where the corresponding microRNA is expressed. As shown in the experimental part, the introduction of target sequences of a microRNA, for example a microRNA expressed in the pancreas or pancreatic islets, upstream of the 3′ UTR leads to specific downregulation of expression in tissues where the corresponding microRNA is expressed, for example the pancreas or pancreatic islets. Furthermore, it is shown that this kind of models can be used for the evaluation of gene therapy approaches.

The highly specific approach is associated with less side effects compared to global and unconditional knock-out and knockdown models. Therefore, plants and non-human animals, particularly non-human animals according to the invention have an increased usefulness to mankind while having fewer undesired side effects.

Compared with existing methods, such as RNAi based methods including overexpression of an artificial RNA (e.g. shRNA), the method of the invention has at least the following benefits and advantages:

Reduction of toxicity problems associated with overexpression of an artificial RNA, including immunological effects and oversaturation of cellular RNA pathways
Reduced likelihood for off-target effects that are widespread with the shRNA approach.
The knock-down effect is naturally confined to the target gene (because the miRNA target site is associated directly with the transgene)
Allowing spatial and temporal control of endogenous gene silencing without the need for any additional genetic elements

Accordingly, the aspects and embodiments of the present invention as described herein solve at least some of the problems and needs as discussed herein.

### Genetically Modified Plants and Non-Human Animals

In a first aspect there is provided a genetically modified plant or non-human animal having reduced expression of an endogenous target gene, wherein the genome of the plant or non-human animal is modified by ectopic integration of at least one copy of a target sequence of a microRNA.

A genetically modified plant or non-human animal having reduced expression of an endogenous target gene as used herein encompasses a genetically modified plant or non-human animal having reduced expression of one, two, three, or more endogenous target genes. Thus, in any embodiments described herein, “an endogenous target gene” may be replaced with “one or more endogenous target genes”.

“Ectopic integration”, as used herein, refers to the integration of a stretch of DNA, particularly at least one copy of a target sequence of a microRNA, at a site other than its usual locus. In other words, a genetically modified plant or non-human animal as described herein is such that the unmodified or wildtype or reference or control or parental genome thereof does not comprise the target sequence of a microRNA at that integration site.

An “endogenous target gene” may be any gene or coding sequence that is naturally present in the plant or non-human animal.

The term “genetically modified” may be understood to refer to any alteration in an organism's genetic material by genetic engineering techniques, i.e. including other steps than merely mating/crossing and selection.

The terms “microRNA” and “target sequence of a microRNA” are further described elsewhere herein.

In any embodiments described herein, “having reduced expression of an endogenous target gene” may be replaced with “wherein an endogenous target gene is silenced”, “wherein an endogenous target gene is knocked down”, “wherein an endogenous target gene is knocked out”, “wherein the expression of an endogenous target gene is prevented”, “wherein the expression of an endogenous target gene is limited”, “wherein the expression of an endogenous target gene is decreased”, or “wherein the expression of an endogenous target gene is inhibited” or similar phrases.

It is understood that “reduced expression” (or silencing, knockdown, and the like) is to be seen relative to an unmodified or wildtype or reference or control or parental plant or non-human animal. In some embodiments, reduced expression (or silencing, knockdown, and the like) may mean at least about 10%, 20%, 30%, 40%, 50%, 60%, 70%, 80%, 90%, 95%, 98%, 99%, or 100% reduction relative to the expression level in an unmodified or wildtype or reference or control or parental plant or non-human animal, preferably at least 60%, more preferably at least 70%, most preferably at least 80%. In some embodiments, reduced expression (or silencing, knockdown, and the like) may mean at least about 10%, 20%, 30%, 40%, 50%, 60%, 70%, 80%, 90%, 95%, 98%, 99%, or 100% reduction in mRNA level relative to the mRNA level in an unmodified or wildtype or reference or control or parental plant or non-human animal, preferably at least 60%, more preferably at least 70%, most preferably at least 80%. In some embodiments, reduced expression (or silencing, knockdown, and the like) may mean at least about 10%, 20%, 30%, 40%, 50%, 60%, 70%, 80%, 90%, 95%, 98%, 99%, or 100% reduction in protein level relative to the protein level in an unmodified or wildtype or reference or control or parental plant or non-human animal, preferably at least 60%, more preferably at least 70%, most preferably at least 80%. In some embodiments, reduced expression (or silencing, knockdown, and the like) may mean that expression is no longer detectable. “Expression” may be assessed as described elsewhere herein, including by the methods described in the examples section.

As used herein, an unmodified or wildtype or reference or control or parental plant or non-human animal or genome may be understood to be a wild type or parental plant or non-human animal from which the genetically modified plant or non-human animal of the invention was derived. In general, a suitable unmodified or wildtype or reference or control or parental plant or non-human animal is any plant or non-human animal having the same or essentially the same genotype as the genetically modified plant or non-human animal of the invention, except for the ectopic integration of the at least one copy of a target sequence of a microRNA.

The term “non-human animal” as used herein includes all multicellular eukaryotic organisms that together form the biological kingdom Animalia, with the exception of humans. The term “plant” as used herein includes all organisms in the biological kingdom Plantae.

In a preferred embodiment, a genetically modified plant or non-human animal as described herein is a genetically modified non-human animal. In some embodiments, a non-human animal is selected from an invertebrate animal and a vertebrate animal, preferably a vertebrate animal.

Invertebrates may be understood to encompass all animal groups not included in the subphylum Vertebrata. Invertebrates include for example arthropods (including insects, arachnids, crustaceans, and myriapods), mollusks (including chitons, snail, bivalves, squids, and octopuses), annelid (including earthworms and leeches), Platyhelminthes, Nematoda, Echinodermata, and cnidarians (including hydras, jellyfishes, sea anemones, and corals). Preferred invertebrates are insects. Vertebrate animals may be selected from the group consisting of: amphibians, reptiles, birds and mammals. Mammals are preferred. In a preferred embodiment, a genetically modified plant or non-human animal as described herein is a mammal such as a rodent, more preferably a rat or mouse, most preferably a mouse.

In some embodiments, a mammal is selected from the group consisting of rodents (including mice, rats, squirrels, prairie dogs, porcupines, beavers, guinea pigs, hamsters, and gerbils), cats, dogs, cows, pigs, horses, sheep, goats and non-human primates (including rhesus macaques, crab-eating macaques, stump-tailed macaques, pig-tailed macaques, squirrel monkeys, owl monkeys, baboons, chimpanzees, marmosets and spider monkeys).

In some embodiments, a genetically modified plant or non-human animal is a model organism, which may be a plant model organism, an invertebrate animal model organism or a vertebrate animal model organism. A model organism is a non-human organism that has been widely studied, usually because it is easy to maintain and breed in a laboratory setting and because it has particular experimental advantages. For example, methods for genetic engineering are usually well-developed in model organisms.

Suitable model organisms which are plants are selected from the group consisting of: Amborella trichopoda, Antheroceros agresis, Arabidopsis thaliana, Arabidopsis halleri, Arabidopsis lyrata, Aquilegia caerulea, Azolla filiculoides, Boechera spp., Brachypodium distachyon, Cardamine hirsute, Ceratodon purpureus, Ceratopteris richardii, Eucalyptus globulus, Eucalyptus grandis, Eutrema salsugineum, Fragaria vesca, Klebsormidium flaccidum, Lemna gibba, Lemna minor, Lotus japonicas, Marchantia polymorpha, Medicago truncatula, Mimulus guttatus, Miscanthus sinensis, Nicotiana benthamiana, Nicotiana tabacum, Oropetium thomameum, Oryza sativa, Panicum virgatum, Petunia×hybrid, Picea abies/Picea glauca, Pinus taeda, Physcomitrella patens, Populus spp. such as Populus trichocarpa, Prunus persica, Phalaenopsis spp., Selaginella moellendorffii, Setaria viridis, Solanum lycopersicum, Sorghum bicolor, Vitis vinifera, Spirodela polyrhiza, and Zea mays. A preferred model organism which is a plant is an Arabidopsis species, preferably Arabidopsis thaliana.

Suitable model organisms which are non-human invertebrate animals are selected from the group consisting of: Amphimedon queenslandica, Arbacia punctulata, Aplysia, Branchiostoma floridae, Caenorhabditis elegans, Caledia captiva, Callosobrochus maculatus, Chorthippis paralluls, Ciona intestinalis, Daphnia spp. such as Daphnia magna and Daphnia pulex, Coelopidae, Diposidae, Drosophila spp. such as Drosophila melanogaster, Eurpymna scolopes, Galleria melonella, Gryllus, bimaculatus, Hydra, Loligo pealei, Lymnaea stagnalis, Macrostomum lignano, Mnemiopsis leidyi, Nematostella vectensis, Oikopleura dioica, Ormia ochracea, Oscarella carmela, Parhyale hawaiensis, Platynereis dumerilii, Podisma spp., Pristionchus pacificus, Scathophaga stercoraria, Schmidtea mediterranea, Strongylocentrotus purpuratus, Symsagittifera roscoffensis, Tribolium castaneum, Trichoplax adhaerens and Tubifex tubifex.

Suitable model organisms which are non-human vertebrate animals are selected from the group consisting of: Ambystoma mexicanum, Anolis carolinensis, Bombina bombina, Bombina variegate, Canis lupus familiaris, Cavia porcellus, Columba livia domestica, Danio rerio, Felis sylvestris catus, Fundulus heteroclitus, Gallus gallus domesticus, Gasterosteus aculeatus, Heterocephalus glaber, Macaca mulatta, Mesocricetus auratus, Mus musculus, Myotis lucifugus, Nothobranchius furzer, Oryzias latipes, Pan troglodytes, Petromyzon marinus, Poecilia reticulate, Rattus norvegicus, Sigmodon hispidus, Taeniopygia guttata, Takifufu rubripes, Xenopus tropicalis and Xenopus laevis. Preferred among these are mammalian model organisms, more preferred are the rat (Rattus norvegicus) and the mouse (Mus musculus), most preferred is the mouse (Mus musculus).

In some embodiments, the ectopic integration site is located:
in the 5′ UTR of the target gene or in the region between the 5′ UTR and the first exon of the target gene; or
in the 3′ UTR of the target gene or in the region between the last exon and the 3′ UTR of the target gene.

An “ectopic integration site” as used herein can be understood as referring to the site in the genome where the at least one copy of a target sequence of a microRNA is integrated.

Downregulation of expression of the endogenous target gene can be obtained regardless of whether the ectopic integration site is located in the 5′ UTR of the target gene (or in the region between the 5′ UTR and the first exon of the target gene); or in the 3′ UTR of the target gene (or in the region between the last exon and the 3′ UTR of the target gene).

In preferred embodiments, the ectopic integration site is located in the 3′ UTR of the target gene or in the region between the last exon and the 3′ UTR of the target gene. More preferably, the ectopic integration site is located in the region between the last exon and the 3′ UTR of the target gene. In some embodiments, the modified genome comprises one, two, three, four, five, six, seven, eight or more copies, preferably two, three or four copies, more preferably two copies of the target sequence of a microRNA.

In some embodiments, a genetically modified plant or non-human animal as described herein does not comprise a selection marker in its genome. In some embodiments, a genetically modified plant or non-human animal as described herein does not comprise an exogenous recombination site in its genome, such as a LoxP site or an Frt site. This will typically be the case when the genetically modified plant or non-human animal is generated according to preferred methods of the invention involving the use of CRISPR-mediated homology-directed repair (HDR) (as described later herein).

A target sequence of a microRNA as described herein is preferably a target sequence of an endogenous microRNA.

A target sequence of a microRNA as described herein includes target sequences for one, two, three, four or more microRNAs. Thus, in some embodiments, the modified genome comprises at least one copy of a target sequence of a first microRNA, and at least one copy of a target sequence of a second microRNA. In some embodiments, the modified genome comprises at least one copy of a target sequence of a first microRNA, at least one copy of a target sequence of a second microRNA, and at least one copy of a target sequence of a third microRNA. In some embodiments, the modified genome comprises at least one copy of a target sequence of a first microRNA, at least one copy of a target sequence of a second microRNA, at least one copy of a target sequence of a third microRNA, and at least one copy of a sequence of a fourth mircroRNA.

Also the target sequence of a second, third and/or fourth microRNA, if present, may be present in two, three, four, five, six or more, preferably two copies.

A genetically modified plant or non-human animal having reduced expression of an endogenous target gene as described herein, includes genetically modified plants or non-human animals having reduced expression of one, two, three or more endogenous target genes. This may be achieved by ectopic integration of at least one copy of a target sequence of a microRNA at a corresponding number of ectopic integration sites.

In some embodiments a genetically modified plant or non-human animal as described herein may have reduced expression of a first and a second endogenous target gene wherein the genome of the plant or non-human animal is modified by ectopic integration of at least one copy of a target sequence of a microRNA at a first ectopic integration site and by ectopic integration of at least one copy of a target sequence of a microRNA at a second ectopic integration site. The first ectopic integration site may be located:
in the 5′ UTR of the first target gene or in the region between the 5′ UTR and the first exon of the first target gene; or
in the 3′ UTR of the first target gene or in the region between the last exon and the 3′ UTR of the first target gene.

Similarly, the second ectopic integration site may be located:
in the 5′ UTR of the second target gene or in the region between the 5′ UTR and the first exon of the second target gene; or
in the 3′ UTR of the second target gene or in the region between the last exon and the 3′ UTR of the second target gene.

In some embodiments a genetically modified plant or non-human animal as described herein may have reduced expression of a first, a second and a third endogenous target gene wherein the genome of the plant or non-human animal is modified by ectopic integration of at least one copy of a target sequence of a microRNA at a first ectopic integration site and by ectopic integration of at least one copy of a target sequence of a microRNA at a second ectopic integration site and by ectopic integration of at least one copy of a target sequence of a microRNA at a third ectopic integration site. The first and second ectopic integration sites may be located as described herein. The third ectopic integration site may be located:
in the 5′ UTR of the third target gene or in the region between the 5′ UTR and the first exon of the third target gene; or
in the 3′ UTR of the third target gene or in the region between the last exon and the 3′ UTR of the third target gene.

MicroRNAs or miRNAs are small non-coding RNA molecules found in plants, animals and some viruses, that may function in RNA silencing and post-transcriptional regulation of gene expression. A target sequence of a microRNA, and similar phrases such as “target sequence binding to a microRNA” or “binding site of a microRNA”, refer to a nucleotide sequence which is complementary or partially complementary to at least a portion of a microRNA.

A portion of a microRNA, as described herein, means a nucleotide sequence of at least four, at least five, at least six or at least seven consecutive nucleotides of said microRNA. The binding site sequence can have perfect complementarity to at least a portion of an expressed microRNA, meaning that the sequences are a perfect match without any mismatch occurring. Alternatively, the binding site sequence can be partially complementary to at least a portion of an expressed microRNA, meaning that one mismatch in four, five, six or seven consecutive nucleotides may occur. Partially complementary binding sites preferably contain perfect or near perfect complementarity to the seed region of the microRNA, meaning that no mismatch (perfect complementarity) or one mismatch per four, five, six or seven consecutive nucleotides (near perfect complementarity) may occur between the seed region of the microRNA and its binding site. The seed region of the microRNA consists of the 5′ region of the microRNA from about nucleotide 2 to about nucleotide 8 of the microRNA.

The portion of a microRNA as described herein is preferably the seed region of said microRNA. Degradation of the messenger RNA (mRNA) containing the target sequence for a microRNA may be through the RNA interference pathway or via direct translational control (inhibition) of the mRNA. This invention is in no way limited by the pathway ultimately utilized by the miRNA in inhibiting expression of the transgene or encoded protein.

It is well-known in the art that there are marked cell-, tissue-, and organ-specific differences in the expression of miRNAs. For example, in mice, about 30% of total noncoding small RNAs are expressed in a tissue specific manner, and about 17% of total noncoding small RNAs are expressed uniquely in a single tissue (Isakova et al. A mouse tissue atlas of small noncododing RNA. PNAS 2020;117(41):25634-25645, incorporated herein by reference). In addition, some miRNAs may have a specific expression pattern over time, optionally in combination with cell-, tissue-, and organ-specific expression. For example, a miRNA may be expressed in specific developmental stages.

For several tens to hundreds of organisms, including both plants and non-human animals, comprehensive miRNA knowledge has been established, including miRNA sequences and information on distribution of expression of each miRNA among different cells, tissues and organs. In addition, considerable knowledge has been established regarding the dynamics of expression over time, e.g. across developmental time. For example, miRBase comprises miRNA sequences from more than 270 organisms across invertebrates, vertebrates and plants. miRBase is the primary public repository and online resource for microRNA sequences and annotation. The miRBase website provides a wide-range of information on published microRNAs, including their sequences, their biogenesis precursors, genome coordinates and context, literature references, deep sequencing expression data and community-driven annotation. miRBase is available at http://www.mirbase.org, described in Kozomara et al. miRBase: from microRNA sequences to function, Nucleic Acids Research, Volume 47, Issue D1, 8 Jan. 2019, Pages D155—D162, incorporated herein by reference.

In particular, for plants, there are several databases including sequences and expression information for miRNAs, such as:
miRBase, available at http://www.mirbase.org, described in Kozomara et al. miRBase: from microRNA sequences to function, Nucleic Acids Research, Volume 47, Issue D1, 8 Jan. 2019, Pages D155—D162, incorporated herein by reference.
PMRD, available at http://bioinformatics.cau.edu.cn/PMRD/, described in Zhenhai Zhang et al. PMRD: plant microRNA database, Nucleic Acids Research, Volume 38, Issue suppl_1, 1 Jan. 2010, Pages D806—D813, incorporated herein by reference.
miRNEST, available at http://rhesus.amu.edu.pl/mirnest/copy/, described in Szczesniak M W, Makalowska I (2014) miRNEST 2.0: a database of plant and animal microRNAs. Nucleic Acids Res. 42:D74-D77, incorporated herein by reference.
miREX, available at http://www.combio.pl/mirex2, described in Zielezinski et al. mirEX 2.0—an integrated environment for expression profiling of plant microRNAs. BMC Plant Biol. 2015 15:144, incorporated herein by reference.
PmiRKB, available at http://bis.zju.edu.cn/pmirkb/, described in Yijun Meng et al. PmiRKB: a plant microRNA knowledge base, Nucleic Acids Research, Volume 39, Issue suppl_1, 1 Jan. 2011, Pages D181—D187, incorporated herein by reference.
PNRD, available at http://structuralbiology.cau.edu.cn/PNRD/index.php, Xin Yi et al. PNRD: a plant non-coding RNA database, Nucleic Acids Research, Volume 43, Issue D1, 28 Jan. 2015, Pages D982—D989, incorporated herein by reference.
MepmiRDB, available at http://mepmirdb.cn/mepmirdb/index.html, described in Dongliang Yu et al. MepmiRDB: a medicinal plant microRNA database, Database, Volume 2019, 2019, baz070, incorporated herein by reference.
PmiREN, available at http://www.pmiren.com, described in Zhonglong Guo et al. PmiREN: a comprehensive encyclopedia of plant miRNAs, Nucleic Acids Research, Volume 48, Issue D1, 8 Jan. 2020, Pages D1114—D1121, incorporated herein by reference.

More information can be found in Liao P, Li S, Cui X, Zheng Y. A comprehensive review of web-based resources of non-coding RNAs for plant science research. Int J Biol Sci 2018; 14(8):819-832, incorporated herein by reference.

For animals, the following databases and publications including sequences and expression information are available:
miRBase, available at http://www.mirbase.org, described in Kozomara et al. miRBase: from microRNA sequences to function, Nucleic Acids Research, Volume 47, Issue D1, 8 Jan. 2019, Pages D155—D162, incorporated herein by reference.
miRNEST, available at http://rhesus.amu.edu.pl/mirnest/copy/, described in Szczesniak M W, Makalowska I (2014) miRNEST 2.0: a database of plant and animal microRNAs. Nucleic Acids Res. 42:D74-D77, incorporated herein by reference.
Isakova et al. A mouse tissue atlas of small noncododing RNA. PNAS 2020;117(41):25634-25645, incorporated herein by reference.
Ludwig, Nicole, et al. Distribution of miRNA expression across human tissues. Nucleic acids research 44.8 (2016): 3865-3877 (also available at https://genome.ucsc.edu/ and https://ccb-web.cs.uni-saarland.de/tissueatlas/), incorporated herein by reference.
RATEmiRs, available at https://connect.niehs.nih.gov/ratemirs/, described in Bushel, P. R., Caiment, F., Wu, H. et al. RATEmiRs: the rat atlas of tissue-specific and enriched miRNAs database. BMC Genomics 19, 825 (2018), incorporated herein by reference.
de Rie, D., Abugessaisa, I., Alam, T. et al. An integrated expression atlas of miRNAs and their promoters in human and mouse. Nat Biotechnol 35, 872-878 (2017), incorporated herein by reference.
Londin, E., Loher, P., Telonis, A. G., Quann, K., Clark, P., Jing, Y., Hatzimichael, E., Kirino, Y., Honda, S., Lally, M., et al. (2015a). Analysis of 13 cell types reveals evidence for the expression of numerous novel primate- and tissue-specific microRNAs. Proc. Natl. Acad. Sci. 112, E1106-E1115, incorporated herein by reference.
McCall, M. N., Kim, M.-S., Adil, M., Patil, A. H., Lu, Y., Mitchell, C. J., Leal-Rojas, P., Xu, J., Kumar, M., Dawson, V. L., et al. (2017). Toward the human cellular microRNAome. Genome Res. 27, 1769-1781, incorporated herein by reference.

All of the microRNAs as well as their sequence and information about their expression across time and in different cells, tissues and organs as disclosed in the above publications and databases is expressly incorporated herein by reference.

In addition to the above, a skilled person could identify further miRNAs including cell-, tissue- and organ-specific miRNAs in any desired plant or non-human animal organism based on available methods and techniques, such as RNASeq. See for example the methods, in particular RNASeq-based methods, described in any of the publications cited above.

In view of the above, the present invention allows a skilled person to exploit the available miRNA knowledge to design genetically modified plant or non-human animals as described herein. Indeed, it is possible to identify desired miRNA target sequences binding miRNAs that are expressed in a specific type of cell, tissue or organ, or during a specific time such as a developmental stage, to generate a genetically modified plant or non-human animal as described herein.

Accordingly, in some embodiments there is provided a genetically modified plant or non-human animal as described herein, wherein the reduced expression is specifically in one or more target cells, tissues and/or organs of an organism and wherein the target sequence is of a microRNA expressed specifically in said one or more target cells, tissues and/or organs.

In some embodiments, there is provided a genetically modified plant or non-human animal as described herein, wherein the reduced expression is in a specific developmental stage.

As described above, a person skilled in the art can identify such suitable miRNAs in the databases and publications referenced above, or based on experimentation.

If a second, third and/or fourth microRNA are additionally present, as described elsewhere herein, each of the first, second, third and/or fourth microRNA may be expressed in the same cell, organ and/or tissue (or developmental stage), or in two, three, four, or more different cells, organs and/or tissues (or developmental stages).

In the context of any embodiment herein, particularly in relation to plants, a cell, tissue or organ may belong to the root system or the shoot system and may belong to the dermal, ground or vascular tissue types. In some embodiments, a cell, tissue or organ may be selected from the group consisting of: roots (including primary roots and secondary roots), leaves (including petiole, blade), buds, stems (including primary stems and secondary stems, nodes and internodes), flowers (including petals, stamens and carpels), fruits, seeds, tubers, rhizomes, cotyledons, and tissues and cells thereof. In some embodiments, a cell, tissue or organ may be selected from the group consisting of: epidermis, parenchyma, collenchyma, sclerenchyma, xylem (including primary xylem and secondary xylem), phloem (including primary phloem and secondary phloem), cambium and cells and tissues thereof.

In the context of any embodiment herein, particularly in relation to non-human animals, a cell, tissue or organ may be selected from the group consisting of: adipose tissue, adrenal glands, anus, appendix, bladder, bones, bone marrow, brain, bronchi, diaphragm, ears, eyes, fallopian tubes, gallbladder, olfactory epithelium, heart, hypothalamus, joints, kidneys, large intestine, larynx, liver, lungs, lymph nodes, mammary glands, mesentery, mouth, nasal cavity, nose, ovary, pancreas, pineal gland, parathyroid glands, pharynx, pituitary gland, prostate, rectum, salivary glands, skeletal muscles, skin, small intestine, spinal cord, spleen, stomach, teeth, thymus gland, thyroid, trachea, tongue, ureters, urethra, uterus, nerves, ligaments, tendons, clitoris, vagina, vulva, cerebellum, placenta, testes, epididymis, vas deferens, seminal vesicles, bulbourethral glands, penis, scrotum, subcutaneous tissue, foramen ovale, arteries, veins, capillaries, lymphatic vessel, tonsils, and interstitium, and tissues and cells thereof. Preferred cells, organs or tissues are: adipose tissue, bone marrow, brain, ears, eyes, heart, hypothalamus, kidneys, large intestine, liver, lungs, lymph nodes, pancreas, prostate, skeletal muscles, skin, small intestine, spinal cord, spleen, stomach and cerebellum, and tissues and cells thereof. More preferred cells, organs or tissues are selected from the group consisting of: muscle, heart, pancreas, brain, kidney, testis, lung, bone marrow, spleen, intestine and liver, and tissues and cells thereof, preferably the pancreas and tissues and cells thereof.

In some embodiments, there is provided a genetically modified plant or non-human animal as described herein, wherein the reduced expression is specifically in the muscle, heart, pancreas, brain, kidney, testis, lung, bone marrow, spleen, intestine and/or liver, and wherein the target sequence is of a microRNA expressed specifically in the muscle, heart, pancreas, brain, kidney, testis, lung, bone marrow, spleen, intestine and/or liver. As described above, a person skilled in the art can identify such suitable miRNAs in the databases and publications referenced above, or based on experimentation.

In some embodiments, the target sequence of a microRNA expressed specifically in the muscle, heart, pancreas, brain, kidney, testis, lung, bone marrow, spleen, intestine and/or liver as described herein is a target sequence of a microRNA selected from Fig. S3 or Dataset S3 in Isakova et al. A mouse tissue atlas of small noncododing RNA. PNAS 2020;117(41):25634-25645, incorporated herein by reference.

In some embodiments, preferably in the context of a murine organism such as a mouse, the target sequence of a microRNA expressed specifically in the muscle, heart, pancreas, brain, kidney, testis, lung, bone marrow, spleen, intestine and/or liver is selected from target sequences of the following microRNAs:
microRNA expressed in the muscle: mir-149, mir-378d, mir-378a, mir-193b, mir-10b, mir-196b, mir-615, mir-169a-1, mir-196a-2, mir-133b, mir-1 a-1, mir-1 a-2, mir-133a-2, mir-133a-1, mir-7068, mir-3061 and mir-299a
microRNA expressed in the heart: mir-499, mir-208a, mir-490, mir-149, mir-378d, mir-378a, mir-133a, mir-206, mir-1, mir-499
preferably, microRNA expressed in the pancreas: mir-200a, mir-96, mir-1839-1, mir-34a, mir-7b, mir-7-1, mir-7-2, mir-184, mir-375, mir-219a-1, mir-574, mir-802, mir-152 and mir-148a, preferably mir-375
microRNA expressed in the brain: mir-299a, mir-376c, mir-134, mir-182, mir-708, mir-let7e, mir-1298, mir-764, mir-879, mir-6540, mir-135b, mir-9-2, mir-9-1, mir-9-3, mir-124a-3, mir-124a-1, mir-124a-2, mir-384, mir-344, mir-488, mir-323, mir-758, mir-138-2, mir-132, mir-137, mir-212, mir-3099, mir-433, mir-487b, mir-667, mir-666, mir-380, mir-128-2, mir-128-1, mir-300, mir-410, mir-485, mir-369, mir-381, mir-543, mir-127, mir-541, mir-1249, mir-338, mir-329, mir-376b, mir-434, mir-136, mir-673, mir-411, mir-540, mir-377, mir-337, mir-379, mir-382, mir-496a, mir-409, mir-154, mir-495, mir-376a, mir-668, mir-129-1, mir-153, mir-598, mir-1224, mir-138-1, mir-219a-2, mir-344-2, mir-129-2, mir-341, mir-431, mir-204, mir-218-2, mir-218-1, mir-670, mir-592, mir-135a-2, mir-135a-1, mir-330, mir-181d, mir-181b-1, mir-181b-2, mir181a-2, mir181a-1, mir-222, mir125b-2
microRNA expressed in the kidney: mir-107, mir-874, mir-190a, mir-3073b, mir-10a, mir-10b, mir-196b, mir-615, mir-196a-1, mir-196a-2
microRNA expressed in the testis: mir-203, mir-147, mir-193b, mir-10b
microRNA expressed in the lung: mir-34b, mir-92b, mir-34c, mir-449a, mir-449c, mir-351
microRNA expressed in the bone marrow: mir-351, mir-223, mir-130b, mir-142, mir-92-2, mir363, mir-20b, mir-106a
microRNA expressed in the spleen: mir-142, mir-92-2, mir-363, mir-20b, mir-106a, mir-150, mir-155
microRNA expressed in the intestine: mir-145a, mir-215, mir-194-1, mir-194-2, mir-31, mir-200a
microRNA expressed in the liver: mir-107, mir-148a, mir-3105, mir-192, mir-122 and mir-1948, mir-152, mir-199a, mir-215, mir-192, mir-194

The sequences of the above microRNAs are known in the art. As an example, in some embodiments, a target sequence of mir-375 has the sequence of SEQ ID NO: 7, or a sequence having 60%, 70%, 80%, 90%, 95%, 98%, 99%, or 100% sequence identity therewith.

A particularly preferred cell, tissue or organ is the pancreas and tissues and cells thereof. A particularly preferred cell is a pancreatic islet cell, preferably a beta cell. Thus, to achieve reduced expression specifically in the pancreas, pancreatic islet cells, and/or beta-cells, microRNAs that are expressed specifically in the pancreas, pancreatic islet cells, and/or beta-cells are particularly preferred.

In some embodiments, provided herein is a genetically modified plant or non-human animal as described herein, wherein the reduced expression is specifically in the pancreas, preferably in the pancreatic islet cells, more preferably in the beta-cells of the pancreas, and wherein the target sequence is of a microRNA expressed specifically in the pancreas, preferably in the pancreatic islet cells, more preferably in the beta-cells of the pancreas. As described above, a person skilled in the art can identify such suitable miRNAs in the databases and publications referenced above, or based on experimentation.

In some embodiments, the target sequence of a microRNA expressed specifically in the pancreas, preferably in the pancreatic islet cells, more preferably in the beta-cells of the pancreas as described herein is a target sequence of a microRNA listed in Fig. S3 or Dataset S3 in Isakova et al. A mouse tissue atlas of small noncododing RNA. PNAS 2020;117(41):25634-25645, incorporated herein by reference. In some embodiments, a microRNA expressed specifically in the pancreas, preferably in the pancreatic islet cells, more preferably in the beta-cells of the pancreas as described herein is selected from the group consisting of: mir-200a, mir-96, mir-1839-1, mir-34a, mir-7b, mir-7-1, mir-7-2, mir-184, mir-375, mir-219a-1, mir-574, mir-802, mir-152 and mir-148a. In a preferred embodiment the target sequence is a microRNA-375 target sequence.

The invention is not limited to any particular target gene or coding sequence. Depending on the desired phenotype (such as a trait, disorder or disease) of the genetically modified plant or non-human animal, a suitable target gene and a suitable organ, tissue and/or cell can be selected.

It follows from the nature of this invention that genes which have a direct connection to a phenotype may be preferred. Such phenotypes are also called monogenic phenotypes and include monogenic traits, monogenic diseases and monogenic disorders. Even more preferred are genes exerting their effects in particular cells, tissues or organs.

In some embodiments, the target gene as described herein is a gene associated with the development of a disease, preferably a monogenic disease. In some embodiments, the target gene as described herein is a gene associated with the development of a disease of the pancreas, preferably a monogenic disease of the pancreas.

In some embodiments, the target gene as described herein encodes a transcription factor. In some embodiments, the target gene as described herein encodes a pancreatic transcription factor, for example as described in Dassaye R, Naidoo S, Cerf M E. Transcription factor regulation of pancreatic organogenesis, differentiation and maturation. Islets. 2016;8(1):13-3, incorporated herein by reference.

In some embodiments, the target gene as described herein is a gene associated with the development of monogenic diabetes, such as maturity-onset diabetes of the young (MODY) and neonatal diabetes, preferably MODY.

A monogenic diabetes as described herein may be selected from the group consisting of: MODY1, MODY2, MODY3, MODY4, MODY5, MODY6, MODY7, MODY8, MODY9, MODY10, MODY11, MODY12, MODY13, MODY14, permanent neonatal diabetes mellitus and transient neonatal diabetes mellitus, preferably MODY1-14, more preferably MODY1 and MODY3.

MODY1 is a MODY associated with mutations in the gene HNF4A (hepatocyte nuclear factor 4A).

MODY2 is a MODY associated with mutations in the gene GCK (glucokinase).

MODY3 is a MODY associated with mutations in the gene HNF1A (hepatocyte nuclear factor 1A).

MODY4 is a MODY associated with mutations in the gene PDX1 (insulin promoter factor-1).

MODY5 is a MODY associated with mutations in the gene HNF1B (hepatocyte nuclear factor 1B).

MODY6 is a MODY associated with mutations in the gene NEUROD1 (neurogenic differentiation 1).

MODY7 is a MODY associated with mutations in the gene KLF11 (Kruppel-like factor 11).

MODY8 is a MODY associated with mutations in the gene CEL (bile salt dependent lipase).

MODY9 is a MODY associated with mutations in the gene PAX4 (paired box gene 4).

MODY10 is a MODY associated with mutations in the gene INS (insulin).

MODY11 is a MODY associated with mutations in the gene BLK (B-lymphocyte tyrosin kinase).

MODY12 is a MODY associated with mutations in the gene ABCC8 (ATP-binding cassette transporter sub-family C member 8).

MODY13 is a MODY associated with mutations in the gene KCNJ11 (Kᵢᵣ6.2).

MODY14 is a MODY associated with mutations in the gene APPL1 (Adaptor protein, phosphotyrosine interacting with PH domain and leucine zipper 1).

Permanent neonatal diabetes mellitus is associated with mutations in the gene ABCC8 (ATP-binding cassette transporter sub-family C member 8), KCNJ11 (Kᵢᵣ6.2) or INS (insulin). Transient neonatal diabetes mellitus is associated with mutations in the gene ABCC8.

In some embodiments, a target gene as described herein is selected from the group consisting of: HNF4A, GCK, HNF1A, PDX1, HNF1B, NEUROD1, KLF11, CEL, PAX4, INS, BLK, ABCC8, APPL1 and KCNJ11, preferably the target gene is HNF1A or HNF4A.

In some embodiments, a target gene as described herein is a hepatocyte nuclear factor (HNF), preferably HNF1A or HNF4A.

In some embodiments, an HNF1A or HNF4A gene as described herein may be any nucleotide sequence encoding a hepatocyte nuclear factor 1A (HNF1A) or hepatocyte nuclear factor 4A (HNF4A). An HNF1A or HNF4A gene as described herein may be an HNF1A or HNF4A gene of any vertebrate non-human animal as described elsewhere herein. In some embodiments, an HNF1A or HNF4A gene as described herein may comprise, consist essentially of or consist of the nucleotide sequence of any one of SEQ ID NOs: 1-6, or a nucleotide sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity therewith.

SEQ ID NO: 1 represents a nucleotide sequence of a human HNF1A. SEQ ID NO: 2 represents a nucleotide sequence of a human HNF4A. SEQ ID NO: 3 represents a nucleotide sequence of a murine HNF1A. SEQ ID NO: 4 represents a nucleotide sequence of a murine HNF4A. SEQ ID NO: 5 represents a nucleotide sequence of a canine HNF1A. SEQ ID NO: 6 represents a nucleotide sequence of a canine HNF4A. SEQ ID NO: 3-6 are preferred, SEQ ID NO's: 3-4 are more preferred.

In some embodiments, a level of sequence identity or similarity as used herein is preferably 70%. Another preferred level of sequence identity or similarity is 80%. Another preferred level of sequence identity or similarity is 90%. Another preferred level of sequence identity or similarity is 95%. Another preferred level of sequence identity or similarity is 99%.

In some embodiments, a genetically modified plant or non-human animal as described herein may be a plant or non-human animal disease model, more preferably a model for a disease associated with or caused by mutations in the target gene.

Preferred diseases herein are monogenic diseases. In some embodiments, a monogenic disease as described herein is a monogenic disease of the pancreas.

In preferred embodiments, a monogenic disease as described herein is a monogenic diabetes, such as a maturity-onset diabetes of the young (MODY) and a neonatal diabetes, preferably MODY. A monogenic diabetes as described herein may be selected from the group consisting of: MODY1, MODY2, MODY3, MODY4, MODY5, MODY6, MODY7, MODY8, MODY9, MODY10, MODY11, MODY12, MODY13, MODY14, permanent neonatal diabetes mellitus and transient neonatal diabetes mellitus, preferably MODY1-14, more preferably MODY1 and MODY3. Genes associated with each of these types of monogenic diabetes are described earlier herein.

In some embodiments, there is provided a non-human animal disease model comprising a genetically-modified non-human animal as described herein, wherein:
the disease is MODY1 and the target gene is HNF4A;
the disease is MODY2 and the target gene is GCK;
the disease is MODY3 and the target gene is HNF1A;
the disease is MODY4 and the target gene is PDX1;
the disease is MODY5 and the target gene is HNF1B;
the disease is MODY6 and the target gene is NEUROD1;
the disease is MODY7 and the target gene is KLF11;
the disease is MODY8 and the target gene is CEL;
the disease is MODY9 and the target gene is PAX4;
the disease is MODY10 and the target gene is INS;
the disease is MODY11 and the target gene is BLK;
the disease is MODY12 and the target gene is ABCC8;
the disease is MODY13 and the target gene is KCNJ11;
the disease is MODY14 and the target gene is APPL1;
the disease is permanent neonatal diabetes mellitus and the target gene is ABCC8, INS and/or KCNJ11; or
the disease is transient neonatal diabetes mellitus and the target gene is ABCC8.

In preferred embodiments, there is provided a non-human animal disease model comprising a genetically-modified non-human animal as described herein, wherein:
the disease is MODY1 and the target gene is HNF4A; or
the disease is MODY3 and the target gene is HNF1A.

In some embodiments, a non-human animal disease model as described herein may display at least one phenotype that is associated with said disease. Phenotypes, as used herein, may be selected from molecular, physiological, histological, morphological and behavioral phenotypes, depending on the phenotypes that are known in the art to be associated with said disease.

A phenotype that is associated with a monogenic diabetes as described herein may be selected from the group consisting of:
hyperglycemia, preferably mild hyperglycemia;
increased fasted glycemia;
reduced glucose tolerance;
reduced insulinemia; and
reduced islet size and/or beta cell mass.

These phenotypes may be assessed using a variety of methods known in the art, including clinical examination and routine laboratory tests. Laboratory tests may include both macroscopic and microscopic methods, molecular methods, radiographic methods such as X-rays, biochemical methods, immunohistochemical methods and others. Suitable methods are known to the person skilled in the art and for example described in the experimental section.

In this context, “decrease”, “increase” or “reduction” means at least a detectable decrease, increase or reduction using an assay known to a person of skill in the art, such as assays as carried out in the experimental part. The decrease, increase or reduction may be a decrease, increase or reduction of at least about 5%, at least about 10%, at least about 20%, at least about 30%, at least about 40%, at least about 50%, at least about 60%, at least about 70%, at least about 80%, at least about 90%, at least about 95% or at least about 100%.

Hyperglycemia and glucose tolerance could be assessed using techniques known to a person of skill in the art, for example as done in the experimental part. An exemplary marker that could be used in this regard is the blood glucose level.

If the target gene is HNF1A, a preferred ectopic integration site is between the last exon and the 3′ UTR. In mice, for example, the last exon of HNF1A is exon 10 (see SEQ ID NO: 3).

If the target gene is HNF4A, a preferred ectopic integration site is between the last exon and the 3′ UTR. In mice, for example, the last exon of HNF4A is exon 10 (see SEQ ID NO: 4).

In some embodiments, there is provided a non-human animal disease model comprising a genetically-modified non-human animal as described herein, wherein the disease is Mitchell-Riley syndrome and the target gene is RFX6 (regulatory factor X6). According to this embodiment, the reduced expression is preferably specifically in the pancreas and/or the kidney and the target sequence is preferably of a microRNA expressed specifically in the pancreas and/or the kidney.

It is clear for a person skilled in the art that the offspring of the genetically modified plant or non-human animal as described herein constitutes an additional aspect of the present invention. Offspring can be obtained by any suitable methods, including sexual and nonsexual reproduction, for example natural mating and crossing, in vitro fertilization, cloning, vegetative propagation, parthenogenesis, and the like. “Offspring” as used herein encompasses all generations of offspring generated from the genetically modified plant or non-human animal of the invention.

In other aspects, there is provided a cell, tissue or organ derived from a genetically modified plant or non-human animal as described herein.

In some embodiments, such cells may be used to prepare immortalized cell lines using conventional techniques known in the art. Thus, in another aspect, the invention provides an isolated cell line derived from the genetically modified plant or non-human animal of the invention. In some embodiments, the cell line is a pancreatic cell line, such as a murine pancreatic cell line, preferably a mouse pancreatic cell line. In some embodiments, the pancreatic cell line is a pancreatic beta cell line.

In some embodiments, the cells derived from a genetically modified plant or non-human animal as described herein may be present in an organoid or an artificial organ, preferably a pancreas organoid or an artificial pancreas. An “organoid” as defined herein is a miniaturized and simplified version of an organ produced in vitro in three dimensions that shows realistic micro-anatomy. The skilled person is able to arrive at such artificial organs and/or organoids using the cell, tissue or organ derived from a genetically modified plant or non-human animal as described herein by applying generally known procedures in the art.

### Methods for Generating Genetically Modified Plants or Non-Human Animals

A genetically modified plant or non-human animal as described herein can be obtained by methods known in the art. More information can be found in handbooks such as Mouse Genetics: Methods and Protocols. Methods in Molecular Biology 2014, Singh and Coppola (Eds.), Springer, ISBN: 978-1-4939-1214-8; Principles of Plant Genetics and Breeding, 2^{nd} Edition (2012), George Acquaah, Wile-Blackwell; From plant genomics to plant biotechnology, 1st edition, 2013, Poltronieri, Burbulis, Fogher (Eds.), Woodhead Publishing; Sambrook and Green, Molecular Cloning. A Laboratory Manual, 4^{th} Edition (2012) Cold Spring Harbor Laboratory Press; Transgenic animal technology: a laboratory handbook, 3rd edition 2014, Carl Pinkert (Ed.), Elsevier; all of which are incorporated herein by reference.

Accordingly, in an aspect there is provided a method for obtaining a genetically modified plant or non-human animal as described herein, comprising:
(a) providing a cell of the plant or non-human animal;
(b) genetically modifying the cell by ectopic integration of the at least one copy of a target sequence of a microRNA in the genome of the cell;
(c) generating an embryo from the cell; and
(d) growing said embryo to form a genetically modified plant or non-human animal.

A cell as recited in step (a) above may be any type of cell. In some embodiments, particularly in the context of non-human animals as described herein, the cell is a pluripotent cell, such as an embryonic stem cell or an induced pluripotent stem cell, or a zygote. Embryonic stem cells can be obtained by suitable methods known in the art, including by isolating them from the inner cell mass of a blastocyst. It is also possible to use induced pluripotent stem cells. More information about induction of pluripotent stem cells can be found in Liu, G., David, B. T., Trawczynski, M. et al. Advances in Pluripotent Stem Cells: History, Mechanisms, Technologies, and Applications. Stem Cell Rev and Rep 16, 3-32 (2020), incorporated herein by reference. Zygotes may be harvested by methods known in the art, for example as described by Cho A, Haruyama N, Kulkarni A B. Generation of transgenic mice. Curr Protoc Cell Biol. 2009;Chapter 19:Unit-19.11, incorporated herein by reference.

In some embodiments, particularly in the context of plants as described herein, the cell is a pluripotent cell. Many plant cells are pluripotent or even totipotent, meaning that most or all cells isolated from a mature plant are suitable and can be used to form a new plant.

Integration of the at least one copy of a target sequence of a microRNA as recited in step (b) may be obtained by any suitable method for genetic modification as known to a person skilled in the art. Step (b) encompasses the introduction of the DNA comprising the at least one copy of a target sequence of a microRNA in the cell of step (a) and the stable integration thereof in the cell's genome.

In some embodiments, particularly in the context of non-human animals as described herein, the DNA comprising the at least one copy of a target sequence of a microRNA is introduced into the cell of step (a) using a nonviral method or a viral method (transduction). Non-viral methods may be selected from the group consisting of chemical-based transfection and non-chemical-based transfection. Chemical-based transfection methods include transfection based on calcium phosphate, cationic polymers such as DEAE-dextran or polyethylenimine (PEI), liposomes (lipofection), fugene and dendrimers. Non-chemical methods include electroporation, cell squeezing, microinjection, sonoporation, optical transfection, protoplast fusion, impalefection, hydrodynamic delivery. In some embodiments, microinjection is preferred. Viral methods utilize the ability of a virus to inject its DNA inside a host cell. Suitable viruses include retrovirus, lentivirus, adenovirus, adeno-associated virus, and herpes simplex virus. For the introduction of DNA comprising the at least one copy of a target sequence of a microRNA into a zygote, microinjection is preferred, for example as described in Cho A, Haruyama N, Kulkarni A B. Generation of transgenic mice. Curr Protoc Cell Biol. 2009;Chapter 19:Unit-19.11, incorporated herein by reference.

In some embodiments, particularly in the context of plants as described herein, the DNA comprising the at least one copy of a target sequence of a microRNA is introduced into the cell of step (a) by electroporation, microinjection, gene gun, or Agrobacterium-mediated transformation.

In some embodiments, stable integration of the DNA comprising the at least one copy of a target sequence of a microRNA may be obtained by homologous recombination. Regions of homology direct the genetic modification to the desired site. In preferred embodiments, the at least one copy of a target sequence of a microRNA is introduced by homology-directed repair (HDR). In preferred embodiments, homology-directed repair (HDR) is CRISPR-mediated HDR. CRISPR-mediated HDR increases the efficiency of standard HDR and relies on the repair of site-specific DNA double-strand breaks (DSBs) induced by RNA-guided endonucleases such as the Cas9 endonuclease. Homology-directed repair (HDR) of these DSBs enables precise editing of the genome by introducing defined genomic changes, including base substitutions, sequence insertions, and deletions (Hsu, P. D., Lander, E. S. & Zhang, F. Development and applications of CRISPR-Cas9 for genome engineering. Cell 157, 1262-1278 (2014), incorporated herein by reference). In some embodiments, step (b) involves the administration of:
a specific gRNA targeting the ectopic integration site;
a donor DNA comprising the target sequence of a microRNA; and
mRNA encoding an RNA-guided endonuclease such as Cas9 mRNA.

In some embodiments, the gRNA has the sequence of SEQ ID NO: 8 or 9, or a sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity therewith. SEQ ID NO: 8 is a sgRNA targeting the region adjacent to exon 10 and the 3′ UTR of the HNF1A and can be used in generating a MODY 3 disease model. SEQ ID NO: 9 is a sgRNA targeting the region adjacent to exon 10 and the 3′ UTR of the HNF4A gene and can be used in generating a MODY1 disease model.

To aid in step (b), a positive selection marker gene may be co-transfected, which gives the cell some selectable advantage, such as resistance towards a certain toxin. If the toxin is added, only those cells with the marker gene (and, hence, the at least one copy of a target sequence of a microRNA) integrated into their genomes will be able to proliferate, while other cells will die. After applying this selective stress (selection pressure) for some time, only the cells with a stable transfection remain and can be cultivated further. Common marker genes are genes encoding resistance against toxins such as geneticin (G418), puromycin, zeocin, hygromycin B and blasticidin S. In some embodiments, a negative selective marker may be used as well, such as a TK gene, the presence of which can be selected against by growing cells in ganciclovir. Negative markers can be used to select for cells that integrated the at least one copy of a target sequence of a microRNA by homologous recombination rather than random insertion.

For step (c), in order to generate an embryo from a cell obtained in step (b), a skilled person may rely on any suitable method that is known in the art.

In some embodiments, particularly in the context of plants as described herein, plant tissue culture techniques allow to grow an embryo and a whole new plant from the cell obtained in step (b). Plant tissue culture is a collection of techniques used to maintain or grow plant cells, tissues or organs under sterile conditions on a nutrient culture medium of known composition. Plant tissue culture relies on the fact that many plant cells have the ability to regenerate a whole plant (i.e., they are pluripotent or totipotent) as described above. More information can be found in Sathyanarayana, B. N. (2007). Plant Tissue Culture: Practices and New Experimental Protocols. I. K. International. ISBN 978-81-89866-11-2; George, Edwin F.; Hall, Michael A.; De Klerk, Geert-Jan, eds. (2008). Plant propagation by tissue culture. (3rd ed.). Springer. ISBN 978-1-4020-5004-6; and Singh, S. K.; Srivastava, S. (2006). Plant Tissue Culture. Campus Book International. ISBN 978-81-8030-123-0; all of which are incorporated herein by reference. In some embodiments, particularly in the context of non-human animals as described herein, step (c) may involve the insertion of the cell obtained in step (b) in a blastocyst to generate an early-stage embryo. In some embodiments, particularly in the context of non-human animals as described herein, step (c) may involve implantation of a microinjected zygote of step (b) into a recipient non-human animal, preferably a pseudo-pregnant recipient non-human animal, for example as described in Cho A, Haruyama N, Kulkarni A B. Generation of transgenic mice. Curr Protoc Cell Biol. 2009;Chapter 19:Unit-19.11, incorporated herein by reference. The zygote will develop into an embryo in the recipient organism.

For step (d), in order to grow the embryo obtained in step (c) into a genetically modified plant or non-human animal, a skilled person may rely on any suitable method that is known in the art.

In some embodiments, particularly in the context of plants as described herein, plant tissue culture techniques allow to grow a plant from the embryo obtained in step (c).

In some embodiments, particularly in the context of non-human animals as described herein, the blastocyst may be implanted into the uterus of a female non-human animal, where they will develop into a new non-human animal. In some embodiments, the embryo that had developed from the zygote implanted into a recipient non-human animal in step (c) may grow further into a genetically modified plant or non-human animal in said recipient non-human animal.

A plant or non-human animal obtained in step (d) described above is typically referred to as the F0 generation. In some embodiments, a method for obtaining a genetically modified plant or non-human animal as described herein may further comprise a step (step (e)) of back-crossing the genetically modified plant or non-human animal obtained in step (d) with unmodified or wildtype or reference or control or parental plant or non-human animal as described herein. Such optional additional step (e) gives rise to a genetically modified plant or non-human animal typically referred to as the F1 generation. The optional additional step (e) may be repeated one or more additional times. Thus, the F1 generation may be backcrossed again with non-genetically modified plant or non-human animal, the resulting progeny may be backcrossed again with unmodified or wildtype or reference or control or parental plant or non-human animal, and so on. Preferably, the genetically modified plant or non-human animal obtained in step (d), i.e. the F0 generation, is backcrossed twice. Such backcrossing as described herein is useful for reducing the number and effects of possible off-target mutations that may have been introduced as a side effect of step (b).

Accordingly, in an aspect there is provided a method for obtaining a genetically modified plant or non-human animal as described herein, comprising:
(a) providing a cell of the plant or non-human animal;
(b) genetically modifying the cell by ectopic integration of the at least one copy of a target sequence of a microRNA in the genome;
(c) generating an embryo from the cell;
(d) growing said embryo into a genetically modified plant or non-human animal; and
(e) back-crossing the genetically modified plant or non-human animal obtained in step (d) with non-genetically modified plant or non-human animal.

Any of the steps (b), (c), (d) and (e) may additionally include a step of genotyping cells, embryos or animals. Genotyping may be used to assist in the selection of cells, embryos or animals in which the at least one copy of a target sequence of a microRNA is introduced at the desired ectopic integration site. Genotyping may be performed by any suitable method known in the art, including restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads. Preferably, genotyping will be performed with PCR. For example, in the experimental section, genotyping was done by PCR using primers of SEQ ID NO: 18 and 19 targeting exon 10 and the 3′ UTR of the HNF4A gene for a MODY1 model, and SEQ ID NO: 20 and 21 targeting exon 10 and the 3′ UTR of the HNF1A gene for a MODY 3 model. Whole-genome sequencing may be used as well, and has the additional advantage that the extent of possible off-target mutations can be assessed.

### Methods and Uses

As described earlier herein, this invention in some aspects relates to a genetically modified plant or non-human animal which is a plant or non-human animal disease model, more preferably a model for a disease associated with or caused by mutations in the target gene. Accordingly, in another aspect, there is provided a method for identifying and/or evaluating therapeutic efficacy of a candidate agent for treating, inhibiting or preventing a disease, comprising administering the candidate agent to a plant or non-human animal disease model, or a cell, tissue or organ derived thereof, as described herein. Preferred diseases are described elsewhere herein.

Candidate agents encompass both existing drugs and new drugs. Existing drugs may be researched for new indications, and may be selected for example from the Drug Repurposing Hub of the Broad Institute, a curated and annotated collection of FDA-approved drugs, clinical trial drugs, and pre-clinical tool compounds with a companion information resource (available et https://clue.io/repurposing, described in Corsello et al. The Drug Repurposing Hub: a next-generation drug library and information resource. Nature Medicine. 23,405-408 (2017), incorporated herein by reference). Candidate agents encompass small molecules as well as biological drugs, including gene therapy.

Candidate agents may be administered as compositions, preferably as pharmaceutical compositions. Such composition comprises the candidate agent and optionally further comprises one or more pharmaceutically acceptable ingredients. As used herein, “pharmaceutically acceptable ingredients” include pharmaceutically acceptable carriers, fillers, preservatives, solubilizers, vehicles, diluents and/or excipients. Accordingly, the one or more pharmaceutically acceptable ingredients may be selected from the group consisting of pharmaceutically acceptable carriers, fillers, preservatives, solubilizers, vehicles, diluents and/or excipients. Such pharmaceutically acceptable carriers, fillers, preservatives, solubilizers, vehicles, diluents and/or excipients may for instance be found in Remington: The Science and Practice of Pharmacy, 23rd edition. Elsevier (2020), incorporated herein by reference.

As described earlier herein, this invention in some aspects relates to a genetically modified plant or non-human animal which is a plant or non-human animal disease model, more preferably a model for a disease associated with or caused by mutations in the target gene, wherein the disease is maturity onset diabetes of the young type 3 (MODY3) and the target gene is HNF1A, or wherein the disease is maturity onset diabetes of the young type 1 (MODY1) and the target gene is HNF4A. Accordingly, in another aspect, there is provided a method for identifying and/or evaluating therapeutic efficacy of a candidate agent for treating, inhibiting or preventing MODY3 or MODY1, comprising administering the candidate agent to a plant or non-human animal which is a MODY3 or MODY1 model as described herein, or a cell, tissue or organ derived thereof.

In some embodiments, a method for identifying and/or evaluating therapeutic efficacy of a candidate agent comprising administering the candidate agent to a plant or non-human animal disease model, or a cell, tissue or organ derived thereof, as described herein, may comprise an additional step of assessing at least one phenotype that is associated with said disease. In some embodiments, assessing at least one phenotype may mean assessing symptoms of disease progression or regression. This can be done by using diagnostic tests known for the skilled person in the art.

Phenotypes and phenotypes that are associated with a monogenic diabetes are described elsewhere herein.

The candidate compound can be administered before, during or after a specific phenotype appears. This may depend, among others, on whether a candidate agent is evaluated for preventive and/or curative efficacy.

### General Information

Unless stated otherwise, all technical and scientific terms used herein have the same meaning as customarily and ordinarily understood by a person of ordinary skill in the art to which this invention belongs, and read in view of this disclosure.

### Pancreas

The term “pancreas” as used herein refers the organ of the digestive system and endocrine system of vertebrates as customarily and ordinarily understood by the skilled person. “Pancreatic islets”, also known as “pancreatic islands” or “islets of Langerhans” refer to the regions of the pancreas that contain its endocrine (hormone-producing) cells as as customarily and ordinarily understood by the skilled person. Pancreatic islets typically comprise alpha-cells, producing glucagon, beta-cells, producing insulin and amylin, delta-cells, producing somatostatin, epsilon-cells, producing ghrelin and PP cells (gamma-cells or F-cells), producing pancreatic polypeptide. Beta-cells are of particurlar importance for maintenance of blood sugar homeostasis.

### Sequence Identity

In the context of the invention, a nucleic acid molecule such as a nucleic acid molecule encoding an HNF1A or HNF4A, is represented by a nucleic acid or nucleotide sequence which encodes a protein fragment or a polypeptide or a peptide or a derived peptide. In the context of the invention, a protein fragment or a polypeptide or a peptide or a derived peptide such as from HNF1A or HNF4A is represented by an amino acid sequence.

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60% amino acid identity or similarity with an amino acid sequence encoded by a nucleotide sequence SEQ ID NO: X.

Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference).

Alternatively, percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.ciov/.

Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, and |
| | Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

### Gene or Coding Sequence

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence (5′ UTR), a coding region and a 3′-nontranslated sequence (3′-end) (3′ UTR) e.g. comprising a polyadenylation- and/or transcription termination site. A chimeric or recombinant gene (such as an HNF1A or HNF4A gene) is a gene not normally found in nature, such as a gene in which for example the promoter is not associated in nature with part or all of the transcribed DNA region. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

A “transgene” is herein described as a gene or a coding sequence or a nucleic acid molecule that has been newly introduced into a cell, i.e. a gene that may be present but may normally not be expressed or expressed at an insufficient level in a cell. In this context, “insufficient” means that although said HNF is expressed in a cell, a condition and/or disease as described herein could still be developed. In this case, the invention allows the over-expression of an HNF. The transgene may comprise sequences that are native to the cell, sequences that naturally do not occur in the cell and it may comprise combinations of both.

### Regulatory Sequence and Promoter

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer.

A “ubiquitous promoter” is active in substantially all tissues, organs and cells of an organism. An “organ-specific” or “tissue-specific” promoter is a promoter that is active in a specific type of organ or tissue, respectively. Organ-specific and tissue-specific promoters regulate expression of one or more genes (or coding sequence) primarily in one organ or tissue, but can allow detectable level (“leaky”) expression in other organs or tissues as well. Leaky expression in other organs or tissues means at least one-fold, at least two-fold, at least three-fold, at least four-fold or at least five-fold lower, but still detectable expression as compared to the organ-specific or tissue-specific expression, as evaluated on the level of the mRNA or the protein by standard assays known to a person of skill in the art (e.g. qPCR, Western blot analysis, ELISA). The maximum number of organs or tissues where leaky expression may be detected is five, six, seven or eight.

Assessment of the ubiquitous or tissue-specific nature of a promoter can be performed by standard molecular toolbox techniques, such as, for example, described in Sambrook and Russel (supra). As a non-limiting example, any expression vector comprising any of the gene construct as described herein, wherein the HNF nucleotide sequence has been replaced by a nucleotide sequence encoding for GFP, can be produced. Cells transduced as described herein can then be assessed for fluorescence intensity according to standard protocols.

### Operably Linked

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame. Linking can be accomplished by ligation at convenient restriction sites or at adapters or linkers inserted in lieu thereof, or by gene synthesis.

### Proteins and Amino Acids

The terms “protein” or “polypeptide” or “amino acid sequence” are used interchangeably and refer to molecules consisting of a chain of amino acids, without reference to a specific mode of action, size, 3-dimensional structure or origin. In amino acid sequences as described herein, amino acids or “residues” are denoted by three-letter symbols. These three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gin) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid.

### microRNA

As used herein, “microRNA” or “mlRNA” or “miR” has its customary and ordinary meaning as understood by one of skill in the art in view of this disclosure. A microRNA is a small non-coding RNA molecule found in plants, animals and some viruses, that may function in RNA silencing and post-transcriptional regulation of gene expression. A target sequence of a microRNA may be denoted as “miRT”. For example, a target sequence of microRNA-1 or miRNA-1 or miR-1 may be denoted as miRT-1.

### Expression

Expression may be assessed by any method known to a person of skill in the art. For example, expression may be assessed by measuring the levels of transgene expression in the transduced tissue on the level of the mRNA or the protein by standard assays known to a person of skill in the art, such as qPCR, RNA sequencing, Northern blot analysis, Western blot analysis, mass spectrometry analysis of protein-derived peptides or ELISA.

Expression may be assessed at any time after administration of the gene construct, expression vector or composition as described herein. In some embodiments herein, expression may be assessed after 1 week, 2 weeks, 3 weeks, 4, weeks, 5 weeks, 6 weeks, 7 weeks, 8 weeks, 9, weeks, 10 weeks, 11 weeks, 12 weeks, 14 weeks, 16 weeks, 18 weeks, 20 weeks, 22 weeks, 24 weeks, 28 weeks, 32 weeks, 36 weeks, 40 weeks, or more.

In the context of the invention, pancreas- and/or pancreatic islet- and/or beta-cell-specific expression refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of HNF, preferably an HNF1A, more preferably an HNF1A isoform a, in the pancreas and/or pancreatic islets and/or beta-cells as compared to other organs or tissues. Other organs or tissues may be the CNS, brain, liver, adipose tissue, skeletal muscle, heart, kidney, colon, hematopoietic tissue, lung, ovary, spleen, stomach, testis and others. Preferably, other organs are the liver and/or the heart. In an embodiment, expression is not detectable in the liver, CNS, brain, adipose tissue, skeletal muscle and/or heart. In some embodiments, expression is not detectable in at least one, at least two, at least three, at least four or all organs selected from the group consisting of the liver, CNS, brain, adipose tissue, skeletal muscle, heart, kidney, colon, hematopoietic tissue, lung, ovary, spleen, stomach and testis. Expression may be assessed as described above.

### Codon Optimization

“Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. For example, to suit the codon preference of mammalians, preferably of murine, canine or human expression hosts. Codon optimization also eliminates elements that potentially impact negatively RNA stability and/or translation (e. g. termination sequences, TATA boxes, splice sites, ribosomal entry sites, repetitive and/or GC rich sequences and RNA secondary structures or instability motifs). In some embodiments, codon-optimized sequences show at least 3%, 5%, 10%, 15%, 20%, 25%, 30%, 40%, 50%, 60%, 70%, 80%, 90%, 100% or more increase in gene expression, transcription, RNA stability and/or translation compared to the original, not codon-optimized sequence.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristic of the invention. In addition, the verb “to consist” may be replaced by “to consist essentially of” meaning that a method as described herein may comprise additional step(s) than the ones specifically identified, said additional step(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The word “about” or “approximately” when used in association with a numerical value (e.g. about 10) preferably means that the value may be the given value (of 10) more or less 1% of the value.

As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. Generation of a MODY3 mouse model. (A) CRISPR/Cas9 strategy to generate MODY3 knock-in (KI) mice. Single guided RNA (sgRNA) was designed to target between exon 10 and 3′ UTR of HNF1α gene to introduce two copies of microRNA 375 target sequence (miRT375), contained in donor DNA, by homology directed repair (HDR). Resultant knock-in allele is represented (bottom). (B) Genotyping of offspring by PCR and subsequent digestion of the PCR amplicon with EcoRV. ND, not digested; WT, wild-type; KI, miRT375 knock-in.

FIG. 2. Downregulation of HNF1A expression levels in islets of MODY3 mice. Gene expression in islets from 14-16-week-old WT/WT (wild-type) and KI/KI (homozygous miRT375 knock-in) (MODY3) mice. Relative expression of Hnf1α (Hepatocyte Nuclear Factor 1-Alpha) in (A) male and (B) female mice. Results are expressed as the mean±SEM. n=5-9. ** p<0.01, *** p<0.001 vs. WT/WT.

FIG. 3. Downregulation of HNF1A production in islets of MODY3 mice. Western-blot analysis of HNF1a protein from islets. A cohort of WT/WT (wild-type), WT/KI (heterozygous) and KI/KI (homozygous) animals were analyzed at 14-16 weeks of age. (A) A representative immunoblot of HNF1α protein and normalized tubulin protein is shown. The graphs showed the densitometric analysis of male (B) and female (C) mice. Results are expressed as the mean±SEM. n=3-5.

FIG. 4. MODY3 mice presented similar HNF1A production in liver than wild-type mice. Western-blot analysis of HNF1α protein from liver of male (A) and female (B) mice. A cohort of WT/WT (wild-type), WT/KI (heterozygous) and KI/KI (homozygous) animals were analyzed at 14-16 weeks of age. Representative immunoblots are shown). The graph shows the densitometric analysis of two different immunoblots (bottom). Results are expressed as the mean±SEM. n=3-6.

FIG. 5. MODY3 Knock-in mice did not exhibit changes in body weight. Body weight evolution of WT/WT (wild-type), WT/KI (heterozygous) and KI/KI (homozygous) from 4 to 14 weeks of age in male (A) and female (B) mice. Results are expressed as the mean±SEM. n=8-12

FIG. 6. MODY3 Knock-in mice presented mild hyperglycemia. Glycemia evolution of WT/WT (wild-type), WT/KI (heterozygous) and KI/KI (homozygous) from 4 to 14 weeks of age in male (A) and female (B) mice. Results are expressed as the mean±SEM. n=8-12.

FIG. 7. Fasted glycemia was increased in MODY3 Knock-in young mice. Fasted glycemia of WT/WT (wild-type) and KI/KI (homozygous) of 6 weeks of age in male (A) and female (B) mice. Results are expressed as the mean±SEM. n=4-16. ** p<0.01, *** p<0.001 vs. WT/WT.

FIG. 8. Fasted glycemia was increased in MODY3 Knock-in adult mice. Fasted glycemia of WT/WT (wild-type) and KI/KI (homozygous) of 12-13 weeks of age in male (A) and female (B) WT/WT and KI/KI mice. Results are expressed as the mean±SEM. n=10-16. ** p<0.01, *** p<0.001 vs. WT/WT.

FIG. 9. MODY3 young mice presented impaired glucose tolerance. Glucose tolerance test was performed after an intraperitoneal injection of glucose (2 g of glucose/kg body weight) at 6 weeks of age in male (A) and female (B) WT/WT (wild-type) and KI/KI (homozygous) mice. Results are expressed as the mean±SEM. n=4-16. * p<0.05, ** p<0.01, *** p<0.001 vs. WT/WT.

FIG. 10. MODY3 adult mice exhibit impaired glucose tolerance. Glucose tolerance test was performed after an intraperitoneal injection of glucose (2 g of glucose/kg body weight) at 12-13 weeks of age in WT/WT (wild-type) and KI/KI (homozygous) male (A) and female (B) mice. Results are expressed as the mean±SEM. n=6-12. ** p<0.01, *** p<0.001 vs. WT/WT.

FIG. 11. MODY3 Knock-in mice presented a reduction of fed serum insulin. Fed serum insulin levels at 14-16 weeks of age in WT/WT (wild-type) and KI/KI (homozygous) male (A) and female (B) mice. Results are expressed as the mean±SEM. n=7-12.

FIG. 12. Reduction of islet size and beta cell mass in adult MODY3 mice. Immunohistochemical detection of insulin in pancreas of 14-16-weeks-old WT/WT (wild-type) and KI/KI (homozygous) male mice. Quantification of (A) islet number, (B) mean islet area (μm²), (C) fold change β-cell mass vs. wild-type group. Results are expressed as the mean±SEM. n=3-4.

FIG. 13. Downregulation of HNF1α target gene expression in adult MODY3 mice. Gene expression in islets from 14-16-week-old WT/WT (wild-type) and KI/KI (homozygous) male mice. Relative expression of Hnfla target genes: L-pk (L-pyruvate kinase), Glut2 (Glucose transporter 2), Nbat (neuroblastoma associated transcript 1), Igf1 (Insulin Like Growth Factor 1), Ins1 (insulin 1), Hnf4a (hepatocyte nuclear factor 4 alpha), Hnf1b (hepatocyte nuclear factor 1 beta), Pdx1 (pancreatic and duodenal homeobox 1), and Hnf3b (hepatocyte nuclear factor 3 beta) in (A) male and (B) female mice. Results are expressed as the mean±SEM. n=6-8. * p<0.05, ** p<0.01, *** p<0.001 vs. WT/WT.

FIG. 14. Generation of a MODY1 mouse model. (A) CRISPR/Cas9 strategy to generate MODY1 knock-in (KI) mice. A single guided RNA (sgRNA) was designed to target between exon 10 and the 3′ UTR of the HNF4A gene to introduce two copies of the microRNA 375 target sequence (miRT375), contained in donor DNA, by homology directed repair (HDR). The wild-type and the resultant knock-in allele is represented. (B) Genotyping of offspring by PCR and subsequent digestion of the PCR amplicon with EcoRV. #47, #49, #50: “horn” homozygous KI (2 fragments after EcoRV digestion: 267 bp, 335 bp); #44: “het” heterozygous KI (WT allele (547 bp) and 2 fragments after digestion (267 bp, 335 bp)); #45, #46, #48, #51: WT (only WT allele: 547 bp).

FIG. 15. Intraductal administration of AAV8 vectors encoding GFP. Nine weeks-old wild-type male mice were intraductally administered with 1×10\hat{ }12 vg/animal of AAV8-RIPI-GFP, AAV8-RIPII-GFP, AAV8-hINS1.9-GFP or AAV8-hINS385-GFP vectors. Gene expression in islets from 11-week-old wild-type mice. Relative expression of GFP in islets and liver. Results are expressed as the mean±SEM. n=6-7. * p<0.005, *** p<0.001 vs. AAV8-RIPI-GFP. ^{$} p<0.05, ^{$$} p<0.01 vs. AAV8-RIPII-GFP. ^{&} p<0.05, ^{&&} p<0.01 vs. AAV8-hINS1.9-GFP.

FIG. 16. Intraductal administration of AAV8 vectors encoding mmHNF1A_a under the control of rat insulin promoters. Nine weeks-old wild-type male mice were intraductally administered with 1×10\hat{ }12 vg/animal of AAV8-RIPI-mmHNF1α_a or AAV8-RIPII-mmHNF1α_a vectors. Wild-type mice intraductally administered with PBS served as controls. Gene expression in islets from 17-week-old wild-type mice. Relative expression of (A) endogenous and AAV-derived Hnf1α (Hepatocyte Nuclear Factor 1-Alpha) gene, or (B) endogenous Hnf1α gene. Results are expressed as the mean±SEM. n=6-7. *** p<0.001 vs. PBS.

FIG. 17. Evaluation of islet number and beta-cell mass in mice treated with AAV8-RIPI-mmHNF1α_a or AAV8-RIPII-mmHNF1α_a vectors. Nine weeks-old wild-type male mice were intraductally administered with 1×10\hat{ }12 vg/animal of AAV8-RIPI-mmHNF1α_a or AAV8-RIPII-mmHNF1α_a vectors. Wild-type mice intraductally administered with PBS served as controls. Immunohistochemical detection of insulin in pancreas of 17-weeks-old mice. Quantification of (A) islet number, (B) percentage of β-cell area relative to pancreas area. Results are expressed as the mean±SEM. n=3. *** p<0.001 vs. PBS.

FIG. 18. Intraductal administration of AAV8 vectors encoding mmHNF1A_a under the control of human insulin promoters. Nine weeks-old wild-type male mice were intraductally administered with 1×10\hat{ }12 vg/animal of AAV8-hINS1.9-mmHNF1α_a or AAV8-hINS385-mmHNF1α_a vectors. Wild-type mice intraductally administered with PBS served as controls. Gene expression in islets from 13-week-old wild-type mice. Relative expression of (A) all endogenous and exogenous Hnf1α (Hepatocyte Nuclear Factor 1-Alpha) gene, or (B) only endogenous Hnf1α gene. Results are expressed as the mean±SEM. n=6-7. *** p<0.001 vs. PBS.

FIG. 19. Evaluation of islet number and beta-cell mass in mice treated with AAV8-hINS1.9-mmHNF1α_a or AAV8-hINS385-mmHNF1α_a vectors. Nine-weeks-old wild-type male mice were intraductally administered with 1×10\hat{ }12 vg/animal of AAV8-hINS1.9-mmHNF1α_a or AAV8-hINS385-mmHNF1α_a vectors. Wild-type mice intraductally administered with PBS served as controls. Immunohistochemical detection of insulin in pancreas of 13-weeks-old mice. Quantification of (A) islet number, (B) β-cell mass. Results are expressed as the mean±SEM. n=3. ** p<0.01 vs. PBS.

FIG. 20. AAV-mediated counteraction of hyperglycemia in MODY3 mice. Eight weeks-old KI/KI (homozygous) mice were intraductally administered with 5×10\hat{ }11 vg/animal of AAV8-hINS385-mmHNF1α_a vectors. WT/WT (wild-type) and KI/KI (homozygous) mice intraductally administered with PBS served as controls. Glycemia evolution of WT/WT, KI/KI and KI/KI treated with AAV8-hINS385-mmHNF1α_a a from 8 to 16 weeks-old male mice. Results are expressed as the mean±SEM. n=3-10. ** p<0.05, *** p<0.001 vs. WT/WT. $$ p<0.01, $$$ p<0.001 vs. KI/KI treated with PBS.

FIG. 21. AAV-mediated improvement of glucose tolerance in MODY3 mice. Glucose tolerance test was performed after an intraperitoneal injection of glucose (1 g of glucose/kg body weight) at 18 weeks of age in male WT/WT (wild-type), KI/KI (homozygous) and KI/KI mice treated with AAV8-hINS385-mmHNF1α_a. Results are expressed as the mean±SEM. n=3-10. * p<0.05, ** p<0.01 vs. WT/WT. $ p<0.05 vs. KI/KI treated with PBS.

FIG. 22. Body weight evolution in MODY3 KI mice treated with AAV8-hINS385-mmHNF1α_a vectors. Eight weeks-old KI/KI (homozygous) mice were intraductally administered with 5×10\hat{ }11 vg/animal of AAV8-hINS385-mmHNF1α_a vectors. WT/WT (wild-type) and KI/KI (homozygous) mice intraductally administered with PBS served as controls. Body weight evolution from 8 to 16 weeks-old male mice of WT/WT, KI/KI and KI/KI mice treated with AAV8-hINS385-mmHNF1α_a. Results are expressed as the mean±SEM. n=3-10.

FIG. 23. MODY3 male adult mice exhibit impaired insulin secretion in vitro. In vitro insulin secretion was evaluated in isolated islets from WT/WT (wild-type) and KI/KI (homozygous) male mice (14-16 weeks of age) incubated with low and high glucose concentrations. Insulin levels were evaluated in medium (A) and isolated islets (B). Results are expressed as the mean±SEM. n=4-6. * p<0.05, ** p<0.01, *** p<0.001 vs. WT/WT.

FIG. 24. MODY3 male adult mice exhibit impaired insulin secretion in vivo. An insulin release test was performed after an intraperitoneal injection of glucose (3 g of glucose/kg body weight) at 15 weeks of age in WT/WT (wild-type) and KI/KI (homozygous) male mice. Results are expressed as the mean±SEM. n=4-6. * p<0.05 vs. WT/WT.

FIG. 25. Increased HNF1A expression levels in islets of MODY3 mice treated with AAV8-hINS385-mmHNF1α_a vectors. Expression levels of Hnf1α (Hepatocyte Nuclear Factor 1-Alpha) were evaluated in islets from 14-16-week-old WT/WT (wild-type), KI/KI (homozygous) and KI/KI mice treated with AAV8-hINS385-mmHNF1α_a vectors by qPCR. Results are expressed as the mean±SEM. n=6-7. ** p<0.01 vs. WT/WT, $$ p<0.01 vs. KI/KI treated with PBS.

FIG. 26. Normalization of HNF1A production in islets of MODY3 mice treated with AAV8-hINS385-mmHNF1α_a vectors. HNF1α protein content was evaluated by Western-blot in islets from 14-16-week-old WT/WT (wild-type), KI/KI (homozygous) and KI/KI mice treated with AAV8-hINS385-mmHNF1α_a vectors. (A) A representative immunoblot of HNF1α protein and the normalized tubulin protein is shown. (B) The histograms depict the densitometric analysis of different immunoblots. Results are expressed as the mean±SEM. n=4. ** p<0.01 vs. WT/WT. &&& p<0.001 vs. KI/KI treated with AAV8-hINS385-mmHNF1α_a.

FIG. 27. AAV treatment increases HNF1α target genes expression. Expression levels of the Hnfla target genes Slc2a2 (encoding for glucose transporter 2, GLUT2), L-pk (L-pyruvate kinase) and Hnf4a (hepatocyte nuclear factor 4 alpha) in islets from 14-16-week-old male WT/WT (wild-type), KI/KI (homozygous) and KI/KI mice treated with AAV8-hINS385-mmHNF1α_a vectors. Results are expressed as the mean±SEM. n=5-7.

FIG. 28. Amelioration of fasted glycemia in MODY3 KI mice treated with AAV vectors. Fasted glycemia of male WT/WT (wild-type), KI/KI (homozygous) and KI/KI mice treated with AAV8-hINS385-mmHNF1α_a vectors at 15 weeks of age. Results are expressed as the mean±SEM. n=15-30. *** p<0.001 vs. WT/WT, $$$ p<0.001 vs. KI/KI treated with PBS.

FIG. 29. Counteraction of hyperglycemia in MODY3 mice treated with a low dose of AAV vectors. Eight-week-old male KI/KI (homozygous) mice were intraductally administered with 10\hat{ }11 vg/animal of AAV8-hINS385-mmHNF1α_a vectors. WT/WT (wild-type) and KI/KI (homozygous) mice intraductally administered with PBS served as controls. Glycemia evolution was monitored for 6 weeks. Results are expressed as the mean±SEM. n=16-52. ** p<0.01, *** p<0.001 vs. WT/WT. && p<0.01, &&& p<0.001 vs. KI/KI treated with AAV8-hINS385-mmHNF1α_a.

FIG. 30. Downregulation of Hnf4a expression levels in islets of MODY1 mice. Relative gene expression of Hnf4a (hepatic nuclear factor 4, alpha) in isolated islets from 12-14-weeks-old male WT/WT (wild-type) and KI/KI (homozygous miRT375 knock-in) MODY1 mice using two different primer pairs (Hnf4a_1, Hnf4a_2). Results are expressed as the mean±SEM. n=4. *** p<0.001, **** p<0.0001 vs. WT/WT.

FIG. 31. MODY1 mice presented similar HNF4A production in liver than wild-type mice. Western-blot analysis of HNF4A protein from liver of male mice. A cohort of WT/WT (wild-type) and KI/KI (homozygous) animals were analyzed at 24 weeks of age. A representative immunoblot is shown. The graph shows the densitometric analysis of the respective immunoblot. Results are expressed as the mean±SEM: n=5-6. ns, not significant.

FIG. 32. MODY1 knock-in mice did not exhibit changes in body weight. Body weight evolution of WT/WT (wild-type) and KI/KI (homozygous) mice from 6-24 weeks of age in male and female mice. Results are expressed as the mean±SEM. n=13-15.

FIG. 33. Downregulation of HNF4A target gene Slc2a2 in adult MODY1 mice. Expression of Slc2a2 (encoding for glucose transporter 2, GLUT2) in isolated islets from 12-14-week-old male WT/WT (wild-type) and KI/KI (homozygous) MODY1 mice. Results are expressed as the mean±SEM. n=4. * p<0.05 vs. WT/WT.

### EXAMPLES

### General Procedures to the Examples

### Generation of MODY3 Mice

MODY3 mice were generated using CRISPR/Cas9 technology. The gRNA, donor DNA, and Cas9 mRNA were pronuclearly microinjected in one-cell mice embryos. After Cas9-mediated double strand break and homologous recombination with the donor DNA, the two copies of miRT375 were introduced between the exon 10 and the 3′ UTR of the mouse HNF1A gene.

### MODY3 Mice Genotyping

Forward (GGACTTGGCCAACAGCTAGT, SEQ ID NO: 20) and reverse (GGAGGAGCAGCAGTGTCAAT, SEQ ID NO: 21) primers targeting exon 10 and the 3′ UTR of the HNF1A gene were used for genotyping of the offspring. PCR reaction generated a 392 bp amplicon that was subsequently digested with the EcoRV restriction enzyme. EcoRV digestion generated fragments of 257 and 80 bp in the WT allele; and of 202, 110 and 80 bp in the allele comprising the two miRT375 copies.

### Generation of MODY1 Mice

MODY1 mice were generated using CRISPR/Cas9 technology. The gRNA, donor DNA, and Cas9 mRNA were pronuclearly microinjected in one-cell mice embryos of the genetic background C57BL/6NCrl. After Cas9-mediated double strand break and homologous recombination with the donor DNA, the two copies of miRT375 were introduced between exon 10 and the 3′ UTR of the mouse HNF4A gene.

### MODY1 Mice Genotyping

Forward (TAGAAGAGCTTTCCCTGGGC, SEQ ID NO: 18) and reverse (GGGGTGAAGAAGTTGAGGGA, SEQ ID NO: 19) primers targeting exon 10 and the 3′ UTR of the HNF4A gene were used for genotyping of the offspring. PCR reaction generated a 602 bp amplicon for the knock-in allele and a 547 bp amplicon for the wild-type allele. Subsequent digestion with EcoRV of the wild-type allele revealed no further fragmentation. Subsequent digestion with EcoRV of the knock-in allele with the two miRT375 copies revealed fragments of 335 and 267 bp.

### Subject Characteristics

Male C57Bl/6J mice and MODY3 mice were used. Mice were fed ad libitum with a standard diet (2018S Teklad Global Diets®, Harlan Labs., Inc., Madison, WI, US) and kept under a light-dark cycle of 12 h (lights on at 8:00 a.m.) and stable temperature (22° C.±2). Mice were weighted weekly after weaning. Blood glucose levels were measured with a Glucometer Elite™ analyzer (Bayer, Leverkusen, Germany). For tissue sampling, mice were anesthetized by means of inhalational anesthetic isoflurane (IsoFlo®, Abbott Laboratories, Abbott Park, IL, US) and decapitated. Tissues of interest were excised and kept at −80° C. or with formalin until analysis. All experimental procedures were approved by the Ethics Committee for Animal and Human Experimentation of the Universitat Autonoma de Barcelona.

C57BL/6NCrl and MODY1 mice were kept under specific-pathogen free (SPF) conditions in individually-ventilated cages systems (IVC). Mice were fed ad libitum with a standard chow diet (Altromin 1314, Altromin Spezialfutter GmbH & Co. KG, Lage, Germany) and kept under a light-dark-cycle of 12 h (lights on at 6:00 am), stable temperature (22° C.±2° C.) and humidity (55%±10%). Body weight was measured weekly using a standard laboratory balance (Sartorius, Germany). For tissue collection, mice were sacrificed by decapitation. All experimental procedures were approved by state ethics committee by the government of Upper Bavaria.

### Recombinant AAV Vectors

Single-stranded AAV vectors of serotype 8 were produced by triple transfection of HEK293 cells according to standard methods (Ayuso, E. et al., 2010. Curr Gene Ther. 10(6):423-36). Cells were cultured in 10 roller bottles (850 cm2, flat; Corning™, Sigma-Aldrich Co., Saint Louis, MO, US) in DMEM 10% FBS to 80% confluence and co-transfected by calcium phosphate method with a plasmid carrying the expression cassette flanked by the AAV2 ITRs, a helper plasmid carrying the AAV2 rep gene and the AAV of serotypes 8 cap gene, and a plasmid carrying the adenovirus helper functions. Transgenes used were: GFP or mouse HNF1A isoform A coding-sequence driven by 1) the rat insulin promoter 1 (RIPI): 2) the rat insulin promoter 2 (RIPII); 3) the human full length insulin promoter (hINS1.9); or 4) a shortened version of the human insulin promoter (hINS385). AAV were purified with an optimized method based on a polyethylene glycol precipitation step and two consecutive cesium chloride (CsCl) gradients.

This second-generation CsCl-based protocol reduced empty AAV capsids and DNA and protein impurities dramatically (Ayuso, E. et al., 2010. Curr Gene Ther. 10(6):423-36). Purified AAV vectors were dialyzed against PBS, filtered and stored at −80° C. Titers of viral genomes were determined by quantitative PCR following the protocol described for the AAV2 reference standard material using linearized plasmid DNA as standard curve (Lock M, et al., Hum. Gene Ther. 2010; 21:1273-1285). The vectors were constructed according to molecular biology techniques well known in the art.

### Retrograde Administration through Pancreatic Biliary Duct

The retrograde injection via the pancreatic biliary duct was conducted as previously described (Jimenez et al., Diabetologia. 2011 May;54(5):1075-86). The animals were anesthetized by an intraperitonial injection of ketamine (100 mg/kg) and xylazine (10 mg/kg). Once the area was shaved and an incision of 2-3 cm was made, the abdomen was opened by an incision through the alba line and an abdominal separator was placed. The bile duct was identified. The liver lobes were separated and the bile duct was clamped in the bifurcation of the hepatic triad to prevent spreading of viral vectors to the liver. A 30 G needle was then inserted in the Vater papilla and advanced retrogrally through the biliary duct. The needle was fixed by clamping the duct at the tip of the intestine to secure its position and prevent the escape of viral vectors into the intestine. The solution of the appropriate dose of viral vectors was slowly injected. One min after the injection, the clip that fixed the needle in place was pulled off and a drop of surgical veterinary adhesive Histoacryl (Braun, TS1050044FP) was applied to the puncture site of the needle. Approximately 2 min later, the clip on the biliary duct was removed and the abdominal wall and skin were sutured. The mice were left on a heating meadow to recover from anesthesia and to prevent heat loss.

### Immunohistochemical and Morphometric Analysis

Tissues were fixed for 24 h in formalin (Panreac Química), embedded in paraffin, and sectioned. Pancreas sections were incubated overnight at 4° C. with guinea pig anti-insulin (1:100; I-8510; Sigma-Aldrich). Rabbit anti-guinea pig coupled to peroxidase (1:300; P0141; Dako) was used as secondary antibody. The ABC peroxidase kit (Pierce) was used for immunodetection, and sections were counterstained in Mayer's hematoxylin. Images were taken at 2× magnification for the pancreatic area and 10× or 20× magnifications for islets using a Nikon Eclipse E800 microscope (Nikon, Tokyo, Japan). Image analysis and quantification of areas in μm² were performed using an image analysis software (analySIS 3.0; Soft Imaging System, Center Valley, PA, EEUU). The percentage of the β-cell area in the pancreas was calculated based on two insulin-stained sections separated by 200 μm by dividing the area of all insulin-positive cells per section by the total pancreatic area of that section. The β-cell mass was calculated by multiplying the pancreas weight by the percentage of β-cell area as previously described (Jimenez et al, Diabetologia. 2011 May;54(5):1075-86).

### Isolation of Pancreatic Islets from MODY3 Mice

The pancreatic islets were extracted by pancreas digestion and subsequent isolation of pancreatic islets. In order to digest the pancreas, mice were sacrificed, the abdominal cavity was exposed and 3 ml of a solution of Liberase (Roche, 0104 mg/ml medium without serum M199 (Gibco-Life Technologies 10012-037)) was perfused to the pancreas via the common bile duct. During perfusion, circulation through the Vater ampoule was blocked by placing a clamp. Once perfused, the pancreas was isolated from the animal and kept on ice before being digested at 37° C. for 19 min. To stop digestion and dilute the Liberase solution, 35 ml of cold medium M199 with 10% serum (Biowest S0250-500) were added and the tube stirred for 30 s to completely disintegrate the tissue. Then, two washes with 30 ml and 10 ml respectively of M199 medium supplemented with serum were done. Then, the solution of disintegrated tissue was filtered (450 mm PGI 34-1800-09) and collected into a new tube. The filtrate with 20 ml of medium with serum was centrifuged (Eppendorf 5810R rotor A-4-62) at 200-230×g for 5 min at 4° C. The supernatant was discarded and after carefully removing all traces of the medium, the pellet was resuspended in 13 ml of Histopaque-1077 (Sigma 10771) and M199 medium without serum was added to a volume of 25 ml avoiding mixing the two phases. Then it was centrifuged (Eppendorf 5810R) at 1000×g for 24 min at 4° C. to obtain the pancreatic islets at the interface between the medium and the Histopaque and thus, they were collected with the pipette. Once isolated, the islets were washed twice with 40 ml of medium with serum and centrifuged at 1400 rpm, 2.5 min at room temperature. In the final wash the pellet with islets was resuspended in 15 ml of M199 medium. In this step, and to help their identification under the microscope, the islets were stained by adding a solution of 200 ml Dithizone to the medium (for 10 ml volume: 30 mg Dithizone (Fluka 43820), 9 ml absolute EtOH, 150 μl NH4OH and 850 μl H20). After 5 min of incubation, islets were transferred to a petri dish and were hand-picked under the binocular microscope.

### Isolation of Pancreatic Islets from MODY1 Mice

The common bile duct was exposed by dislocating the gut and liver. A bulldog microclamp (Roboz Surgical Instruments Co., Inc., Gaithersburg, MD, USA) was placed on the Ampulla of Vater, the site where the common bile duct enters the duodenum. A 30 G1/2 needle (Braun, Germany) was then used to enter the common bile duct and 3-4 ml of collagenase solution was perfused into the pancreas. The pancreas was then removed from the cadaver and immediately placed on ice in a 15 ml reaction tube containing 3.5 ml collagenase solution for a maximum of 60 min. The pancreas was further digested in a water bath at 37° C. for 15 min with a gentle shaking step after 7.5 min. Next, 10 ml of ice-cold G-solution was added to stop the reaction, then samples were centrifuged at 290×g for 2 min at RT. The supernatant was carefully decanted. Using an additional 10 ml of G-solution, the remaining pancreatic digest was dissolved by repeated vigorous pipetting. The solution was filtered through a metal mesh (pore size approx. 1 mm) into a 50 ml reaction tube in order to remove larger undigested pieces. Another 10 ml of G-solution, used to rinse the 15 ml tube, was also filtered through the metal mesh. Finally, the metal mesh was rinsed with a further 20 ml of G-solution to avoid loss of islets. The complete filtrate was centrifuged at 290×g for 2 min at RT. After decanting the supernatant, the pellet was resuspended in 5.5 ml of Optiprep-RPMI solution. This resuspension was slowly pipetted along the wall into a new 15 ml tube with 2.5 ml Optiprep-RPMI, creating a gradient, which was then overlaid with 6 ml of G-solution to obtain a third layer. The samples were allowed to incubate an additional 10 min at RT to improve gradient formation before centrifuging at 290×g for 15 min at RT with an adjusted slow acceleration and without break to avoid mixing of the gradients. Islets accumulate between the second and the third layer of the gradient. They were carefully collected with a serological pipet and then filtered through a 70 μm cell strainer to remove any remaining acinar tissue. Islets were captured from the cell strainer by turning the strainer and rinsing it with G-solution into an untreated suspension culture dish. The islets were then picked by hand under a stereomicroscope and placed in a new suspension culture dish with 12 ml islet culture medium (a maximum of 60-80 islets per dish were allowed). Islets were left to rest and recover overnight in an incubator at 37° C. with 5% CO₂ infusion and humidified air before a subsequent islet lysis, RNA isolation or protein isolation was carried out.

### G-Solution

500 ml HBSS (Life technologies #14025092)+5 ml antibiotic-antimycotic solution (Sigma Aldrich)+5 g BSA; sterile filtered

### Collagenase Solution

1 mg/ml Collagenase P (Roche, Germany) in 8 ml G-solution; freshly prepared

### RPMI Medium

445 ml RPMI 1640 medium with UltraGlutamine (Lonza)+5 ml antibiotic-antimycotic solution (Sigma Aldrich)+50 ml FBS (Gibco)

### 10% RPMI

5 ml RPMI medium+45 ml G-solution

### Optiprep

20 ml OptiPrep™ Density Gradient (Sigma Aldrich)+9.7 ml DBPS (Lonza)+0.3 ml HEPES (Lonza)

### Optiprep-RPMI Solution

5 ml Optiprep+3 ml 10% RPMI; freshly prepared

### In Vitro Glucose Stimulated Insulin Secretion

After islet isolation, islets were cultured O/N at 37° C. in RPMI 1640 medium (11 mM glucose), supplemented with 1% BSA, 2 mM glutamine, and penicillin/streptomycin in an atmosphere of 95% humidified air, 5% CO2, to allow recovery from islet isolation stress. Next, 120 islets of similar size isolated from mice of each experimental group were washed in KRBG30 buffer twice and then were handpicked and seeded in a 6-well plate containing KRB G30 for pre-culture during 2 hours at 37° C. in an atmosphere of 95% humidified air, 5% CO2. Then, 150 ul of KRB G30 (low glucose) or KRB G300 (high glucose) were loaded in a 96-well plate (5 wells per condition). After 2 hours, 20 pre-cultured islets per well were loaded in the new 96-well plate containing low or high glucose medium and were incubated during 1 hour at 37° C. After this incubation, medium and (120 μl/well) islets were collected separately. Medium was subsequently stored at −80° C. After collection of islets, acetic acid lysis buffer was added and the mixture was frozen O/N at −80° C. For islet lysis, islets and acetic acid were boiled at 100° C. for 10 min, then spined at 4° C. for 10 min at 12000 rpm. The supernatant was collected and stored at −80° C. Insulin content in islets and insulin concentration in culture medium were measured by ELISA.

### RNA Analysis in MODY3 Mice

Total RNA was obtained from islets or liver by using Tripure isolation reagent (Roche Diagnostics Corp., Indianapolis, IN, US), and RNAeasy Microkit (Qiagen NV, Venlo, NL) for islets and RNeasy Tissue Minikit (Qiagen NV, Venlo, NL) for liver. In order to eliminate the residual viral genomes, total RNA was treated with DNAsel (Qiagen NV, Venlo, NL).

The concentration and purity of the obtained RNA was determined using a device Nanodrop (ND-1000, ThermoScientific). For RT-PCR, 1 μg of RNA samples was reverse-transcribed using Transcriptor First Strand cDNA Synthesis Kit (04379012001, Roche, California, USA). Real-time quantitative PCR was performed in a SmartCyclerII® (Cepheid, Sunnyvale, USA) using EXPRESS SYBRGreen qPCR supermix (Invitrogen™, Life Technologies Corp., Carslbad, CA, US). Data was normalized with Rplp0 values and analyzed as previously described (Pfaffl, M., Nucleic Acids Res. 2001; 29(9):e45).

Primer pairs

| Gene | forward primer | reverse primer |
|---|---|---|
| Rplp0 | TCCCACCTTGTCTCCAG TCT | ACTGGTCTAGGACCCGAGAAG |
| | (SEQ ID NO: 22) | (SEQ ID NO: 23) |
| L-PK | GTTTCTTGGGCAACAGGAAG | AGGAGGCAAAGATGATGTCC |
| | (SEQ ID NO: 24) | (SEQ ID NO: 25) |
| HNF4a | AGATTGACAACCTGCTGCAG | TGCCCATGTGTTCTTGCATC |
| | (SEQ ID NO: 26) | (SEQ ID NO: 27) |
| HNF1a | TGTCACAGCACCTCAACAAG | TGTGGGCTCTTCAATCAGTC |
| | (SEQ ID NO: 28) | (SEQ ID NO: 29) |
| Slc2a2 | ATC CCT TGG TTC ATG | AAT TGC AGA CCC AGT |
| | GTT GC | TGC TG |
| | (SEQ ID NO: 30) | (SEQ ID NO: 31) |

### RNA Analysis in MODY1 Mice

RNA was isolated from overnight-resting islets using the RNeasy Micro kit (Qiagen, Germany) following the manufacturer's instructions. RNA concentration was measured using a NanoPhotometer® device (Implen, Germany). For cDNA synthesis, RNA was reverse-transcribed using SuperScriptIV (Thermo Fisher) following the manufacturer's instructions. Finally, cDNA was adjusted to a concentration of 2.5 ng/μl. Quantitative real-time PCR (qRT-PCR) was performed in a LightCycler® 480 device (Roche, Germany) using the QuantiFast SYBR Green PCR Kit (Qiagen, Germany) following the manufacturer's instructions and with 0.5 ng cDNA per 20 μl reaction in 384-well plates. Results were normalized to house-keeping genes Atp5b and Rpl13a and analyzed using the Livak ΔΔCt method (Methods 25(4), 402-408).

Primer pairs

| Gene | forward primer | reverse primer |
|---|---|---|
| Atp5b | GGTTTGACCGTTGCTGA | TAAGGCAGACACCTCTGAGC |
| | ATAC | (SEQ ID NO: 33) |
| | (SEQ ID NO: 32) | |
| Rpl13a | TGAAGCCTACCAGAAAG | GCCTGTTTCCGTAACCTCAA |
| | TTTGC | (SEQ ID NO: 35) |
| | (SEQ ID NO: 34) | |
| Hnf4a_1 | GTTCTGTCCCAGCAGAT | CTTCTTTGCCCGAATGTCGC |
| | CACC | (SEQ ID NO: 37) |
| | (SEQ ID NO: 36) | |
| Hnf4a_2 | CGTGCTGCTCCTAGGCA | CATCGAGGATGCGGATGGAC |
| | ATG | (SEQ ID NO: 39) |
| | (SEQ ID NO: 38) | |
| Slc2a2 | GGGGACAAACTTGGAAG | TGAGGCCAGCAATCTGACTA |
| | GAT | (SEQ ID NO: 41) |
| | (SEQ ID NO: 40) | |

### Hormone Detection

Insulin concentrations were determined by Rat Insulin ELISA sandwich assay (90010, Crystal Chem INC. Downers Grove, IL 60515, USA).

### Glucose Tolerance Test

Awake mice were fasted overnight (16 h) and administered with an intraperitoneal injection of glucose (1 or 2 g/kg body weight). Glycemia was measured in tail vein blood samples at the indicated time points.

### In Vivo Insulin Release Test

Awake mice were fasted overnight (16 h) and administered with an intraperitoneal injection of glucose (3 g/kg body weight). Venous blood was collected from tail vein in tubes at the indicated time points and immediately centrifuged to separate serum, which was used to measure insulin levels.

### Western Blot Analysis in Samples from MODY3 Mice

Islets or liver were homogenized in Lysis Buffer. Proteins were separated by 10% SDS-PAGE, and analyzed by immunoblotting with rabbit monoclonal anti-HNF1A (D7Z2Q; Cell signaling) and rabbit polyclonal anti-α-tubulin (ab4074; Abcam) antibodies. Detection was performed using ECL Plus detection reagent (Amersham Biosciences).

### Western Blot Analysis in Samples from MODY1 Mice

Liver tissue was dissected into small pieces and protein was extracted using a Precellys Evolution instrument equipped with a Cryolys Evolution cooling unit (Bertin GmbH, Germany) following the manufacturer's instructions. Protein concentrations were determined using the Pierce BCA Assay Kit (Thermo Fisher) according to the manufacturer's instructions. For Western blot analysis, reduced samples (40 μg protein per sample) were prepared using BOLT reagents (Life technologies) following the manufacturer's instructions. Denatured samples were loaded onto BOLT 4-12% gradient gels (Life technologies). The Chameleon Duo Ladder (LI-COR Biotechnology GmbH, Germany) was used to visualize protein separation during electrophoresis and to estimate the molecular weight of proteins. After transfer to nitrocellulose membranes (Life technologies), immunodetection was performed with rabbit monoclonal anti-HNF4A (ab199431, Abcam) and rabbit polyclonal anti-α-tubulin (ab4074, Abcam). Fluorophore-conjugated secondary antibodies (926-32211, LI-COR) were used. Analysis was performed using an Odyssey Infrared Imaging System (LI-COR) followed by densitometric quantification using the Studio Lite Ver5.2 software (LI-COR).

### Statistical Analysis

All values are expressed as mean±SEM. Differences between groups were compared by Student's t-test. Differences were considered significant at p<0.05.

### Example 1. Generation of the New MODY3 Mouse Model

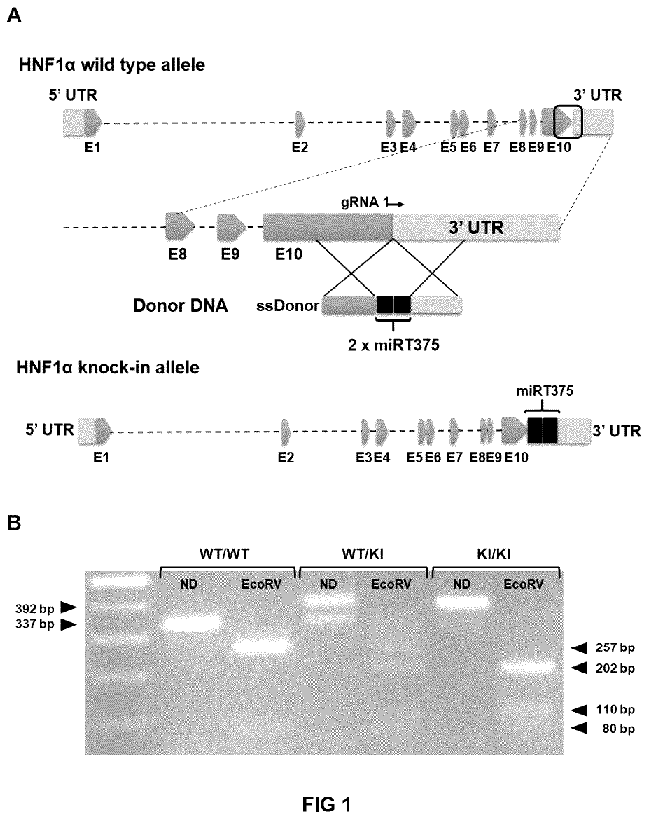

A new β-cell specific mouse model for MODY3 by means of the CRISPR/Cas9 technology was generated. To preclude production of HNF1A specifically in beta-cells, we introduced two copies of the target sequence for the beta-cell specific miRNA375 (miRT375) (SEQ ID NO: 7) upstream the 3′ UTR of the HNF1A gene. Specifically, a single guided RNA (sgRNA) (SEQ ID NO: 8) was designed to target the region adjacent to exon 10 and the 3′ UTR of the HNF1A gene to introduce two copies of the microRNA 375 target sequence (miRT375), contained in DNA donor, by homology directed repair (HDR) (FIG. 1A). miRNAs are small non-coding RNAs that bind specifically to certain mRNAs preventing their translation. Incorporation of target sequences of tissue-specific miRNAs in expression cassettes has been widely used in gene therapy approaches to de-target transgene expression from undesired tissues (Jimenez, V. et al. (2018) EMBO Mol Med 10(8):8791) but to the best of our knowledge nobody has used this approach to generate disease animal models.

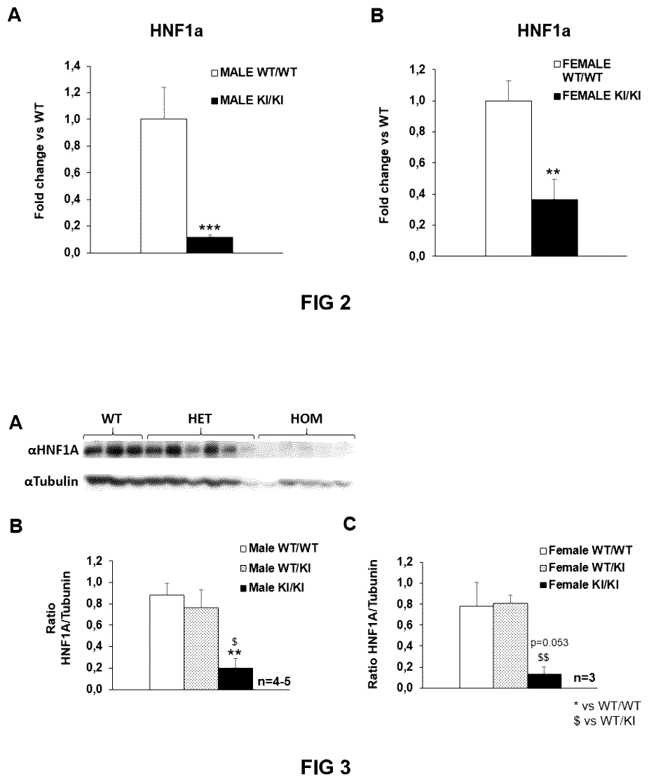

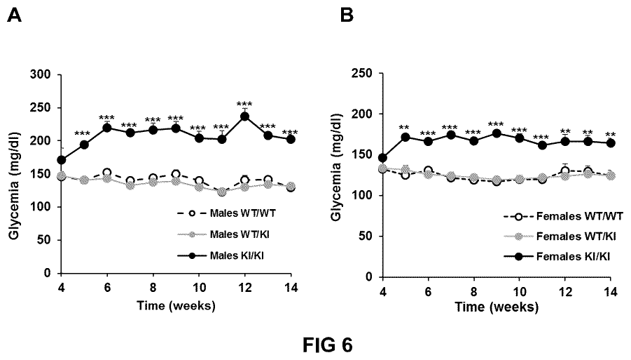

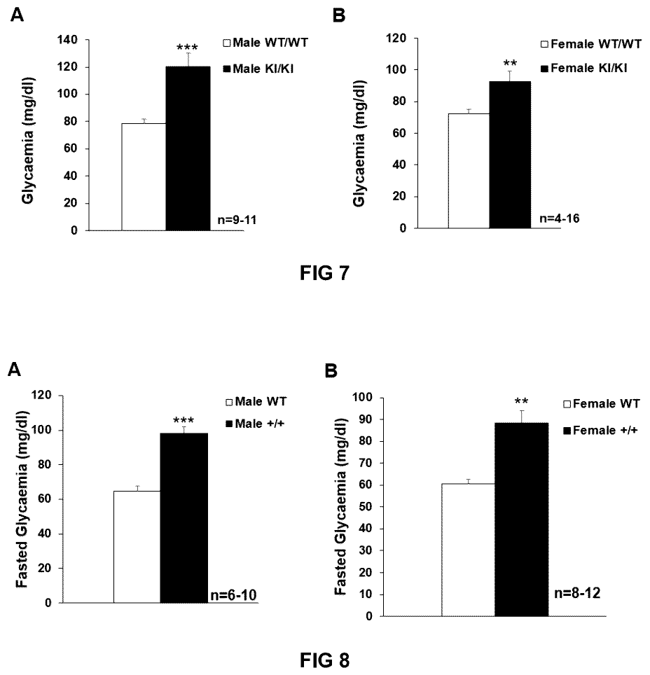

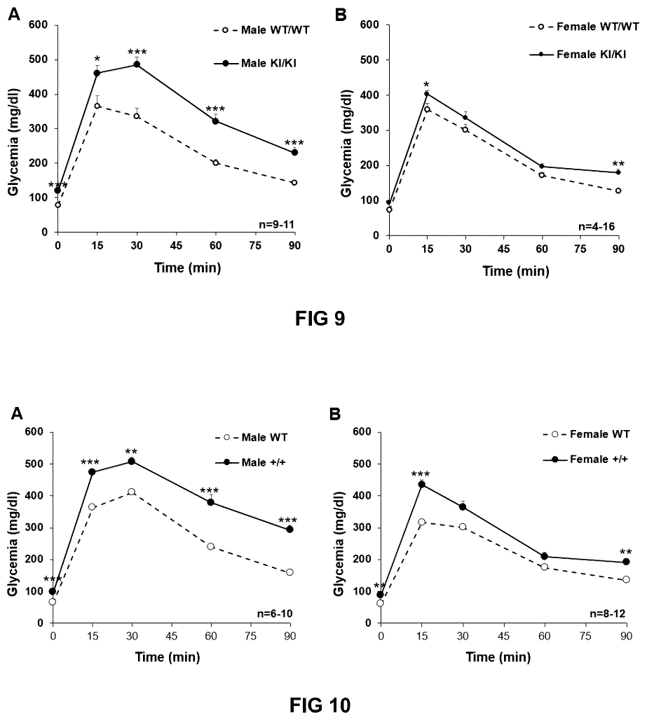

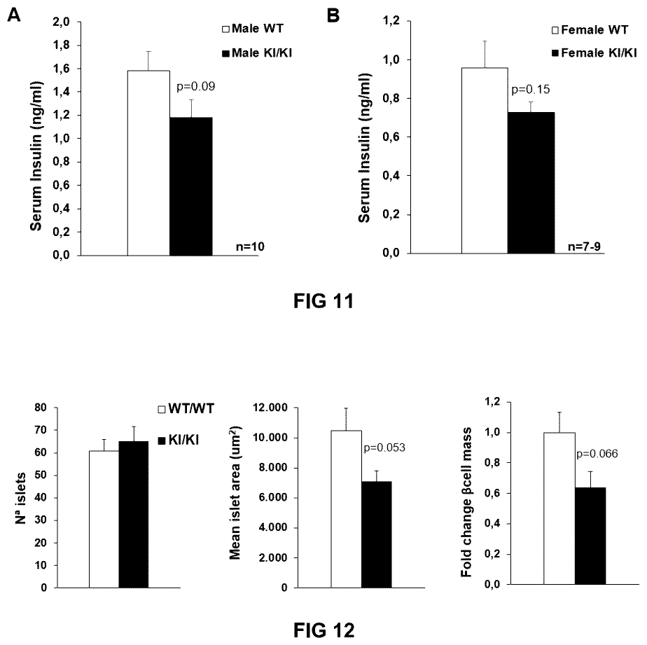

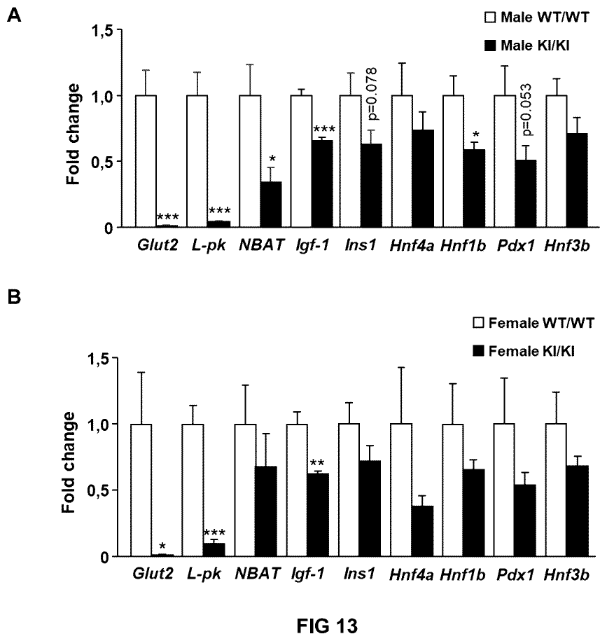

The specific gRNA, the donor DNA, and the Cas9 mRNA were pronuclearly microinjected into one-cell embryos that were subsequently transferred into recipient female mice. F0 generation was genotyped by PCR analysis using specific primers located in the flanking sequences of the knock-in site. Next, the PCR products were digested with EcoRV, leading to different patterns depending on the mice genotype (FIG. 1B). Knock in (KI) mice were backcrossed with control (C57BL6) mice in order to segregate possible CRISPR/Cas9 off-target mutations. Heterozygous mice from the F1 generation were mated again with new control (C57BL6) mice to further segregate off-targets and obtain the F2 generation. F2 heterozygous mice were mated between each other to generate the F3 in which phenotyping of the model was performed. The most important results were:
Specific downregulation of HNF1α expression and production in islets (FIGS. 2, 3 and 4)
Maintenance of body weight (FIG. 5)
Sustained mild hyperglycemia (FIG. 6)
Increased fasted glycemia in young and adults (FIGS. 7 and 8)
Reduced glucose tolerance both in young and adults (FIGS. 9 and 10)
Reduced insulinemia (FIG. 11)
Reduced islet size and beta cell mass (FIG. 12)
Downregulation of HNF1α target genes expression in islets (FIG. 13)

### Example 2. Downregulation of HNF1A Expression and Production Levels in Islets from MODY3 Mice

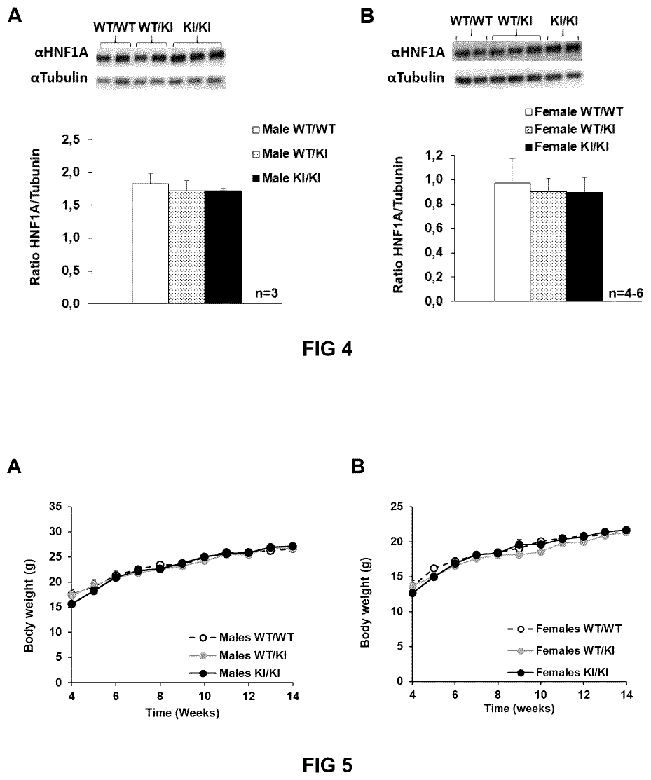

HNF1A expression and protein levels were analyzed in islet samples from 14 to 16-week-old MODY3 mice. Homozygous MODY3 male and female mice showed markedly reduced HNF1A expression levels and HNF1A protein content in islets (=80% reduced HNF1A protein production) (FIGS. 2 and 3). No changes in HNF1A protein content were observed in the liver of MODY3 male and female mice (FIG. 4).

### Example 3. MODY3 Mice Exhibited Mild Hyperglycemia and Impaired Glucose Tolerance

Body weight follow-up demonstrated that wild-type, heterozygous and homozygous MODY3 mice showed similar body weight (FIG. 5). Monitoring of blood glucose levels revealed that, similarly to patients, both male and female homozygous MODY3 mice were mildly hyperglycemic under fed and fasted conditions (FIGS. 6-8).

Moreover, male and female MODY3 mice showed impaired glucose tolerance in comparison with WT mice at young and adult ages (FIGS. 9-10). The diabetic phenotype was more exacerbated in male than female MODY3 mice.

### Example 4. MODY3 Mice Showed Decreased Beta-Cell Mass and Insulinemia

To further evaluate the pancreas phenotype in MODY3 mice, pancreatic sections were immunostained against insulin and morphometric analyses were performed. No striking differences in islet morphology and number of islets were detected between MODY3 and WT mice (FIG. 12A). Nevertheless, MODY3 mice showed reduced mean islet area (FIG. 12B) and β-cell mass in comparison to WT mice (FIG. 12C). In agreement, both male and female homozygous MODY3 mice showed reduced insulinemia (FIG. 11). Thus, the pancreas phenotype of homozygous MODY3 mice resembles that of MODY3 patients, with defects in β-cells and insulopenia (Sanchez Malo, M. J. et al. (2019) Endocrinol Diabetes Nutr;66(4):271-272.).

### Example 5. MODY3 Mice Showed Downregulation of HNF1A Target-Genes and β-Cell Transcriptional Regulatory Network

In pancreatic β-cells, HNF1A has been reported to regulate expression of insulin and β-cell transcription factors as well as expression of proteins involved in glucose transport and metabolism and mitochondrial function, all of which are involved in insulin secretion (Fajans, S. S. et al. (2001). N. Engl. J. Med., 345, 971-80). Both male and female MODY3 mice showed markedly reduced expression of all HNF1A gene targets examined (FIG. 13).

Altogether, a new β-cell specific MODY3 mouse model that faithfully mimics the clinical phenotype of MODY3 patients has been developed. This new mouse model represents a useful tool to assess novel treatment strategies for MODY3.

It is pointed out that neither MODY3 nor MODY1 mice experienced any significant suffering, as evidenced by our results. Their body weight is similar to that of control mice and no increased mortality has been observed. Any possible very mild suffering is limited to displaying mild hyperglycemia.

### Example 6. Generation of the New MODY1 Mouse Model

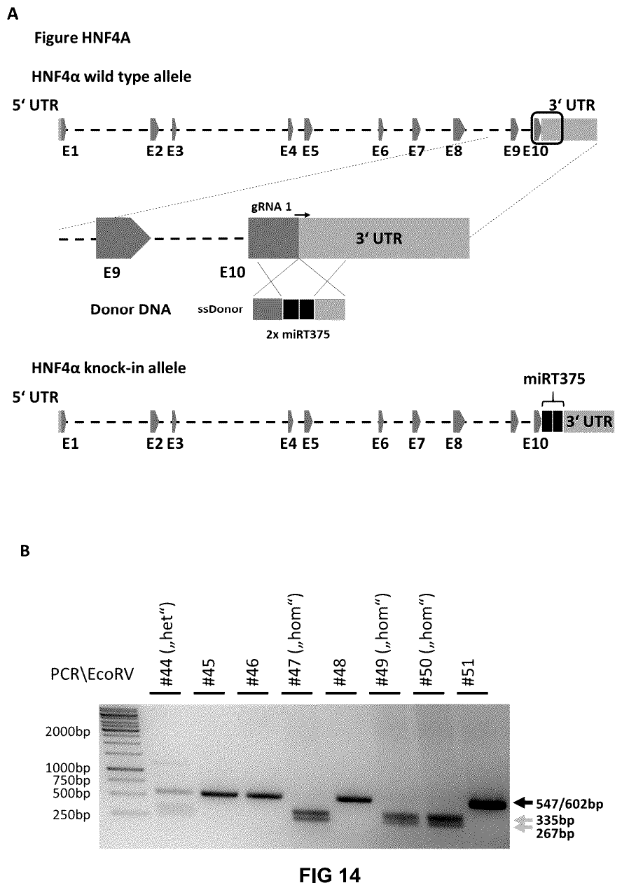

A new beta-cell specific mouse model for MODY1 by means of the CRISPR/Cas9 technology was generated. To preclude production of HNF4A specifically in beta-cells, we introduced two copies of the target sequence for the beta-cell specific miRNA375 (miRT375) (SEQ ID NO: 7) upstream the 3′ UTR of the HNF4A gene. Specifically, a single guided RNA (sgRNA) (SEQ ID NO: 9) was designed to target the region adjacent to exon 10 and the 3′ UTR of the HNF4A gene to introduce two copies of the microRNA 375 target sequence (miRT375) and a new EcoRV restriction site (to be used for genotyping of offspring), contained in DNA donor, by homology directed repair (HDR) (FIG. 14A).

The specific gRNA, the donor DNA, and the Cas9 mRNA were pronuclearly microinjected into one-cell C57BL/6NCrl embryos that were subsequently transferred into recipient female mice. The F0 generation was genotyped by PCR analysis using specific primers located in the flanking sequences of the knock-in site. Next, the PCR products were digested with EcoRV, leading to different patters depending on the mice genotype (FIG. 14B). Knock in (KI) mice were backcrossed with control (C57BL/6NCrl) mice in order to segregate possible CRISPR/Cas9 off-target mutations. Heterozygous mice from the F1 generation were mated again with new control (C57BL/6NCrl) mice to further segregate off-targets and obtain the F2 generation. F2 heterozygous mice were mated between each other to generate the F3 generation, which will be used for phenotyping of the model.

### Example 7. Selection of Beta-Cell Specific Promoter to Drive Expression of HNF1A

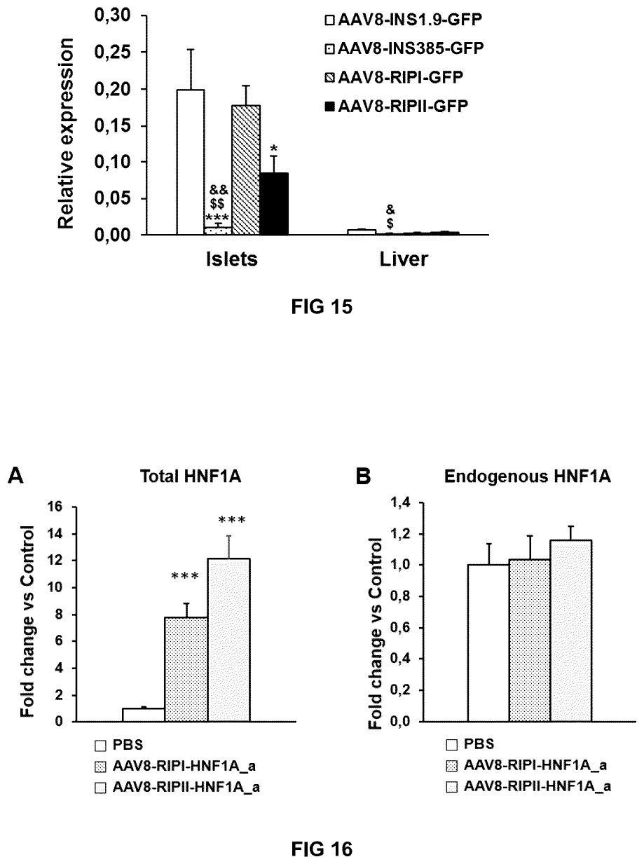

The MODY3 mouse model developed in Example 1 was used to design a suitable gene therapy approach. First, to select the most appropriate beta-cell specific promoter, AAV8 vectors encoding GFP under the control of four candidate promoters were generated. The selected promoters were the rat insulin promoter I (RIPI, SEQ ID NO: 10), rat insulin promoter II (RIPII, SEQ ID NO: 11), the full-length human insulin promoter (hINS1.9, SEQ ID NO: 12), and a 385 by fragment of the human insulin promoter (hIns385, SEQ ID NO: 13). Expression cassettes encoding GFP under the control of either RIPI, RIPII, hINS1.9 or hIns385 promoters and flanked by the inverted terminal repeats (ITRs) of AAV2 were generated. AAV8-GFP vectors (AAV8-RIPI-GFP, AAV8-RIPII-GFP, AAV8-hINS1.9-GFP and AAV8-hIns385-GFP) were produced by triple transfection in HEK293 cells. To evaluate the strength of the promoters and beta-cell specificity of the RIPI, RIPII, hINS1.9 and hIns385 promoters, wild type mice were administered intraductally with AAV8-RIPI-GFP, AAV8-RIPII-GFP, AAV8-hINS1.9-GFP or AAV8-hIns385-GFP vectors. Although all vectors promoted specific GFP overexpression in islets (FIG. 15), RIPI, RIPII and hINS1.9 mediated higher GFP expression levels in islets than the hIns385 promoter.

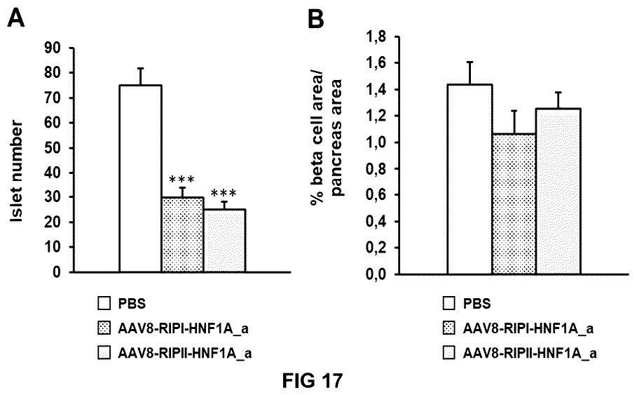

First, expression cassettes encoding the Mus musculus hepatocyte nuclear factor 1A isoform A (HNF1A_a) under the control of either RIPI or RIPII promoters and flanked by the inverted terminal repeats (ITRs) of AAV2 were generated (SEQ ID NO: 14 and 15). AAV8 vectors (AAV8-RIPI-HNF1A_a and AAV8-RIPII-HNF1A_a) were produced by triple transfection in HEK293 cells. To evaluate whether RIPI and RIPII were able to mediate HNF1A_a expression in beta-cells and to assess if this overexpression was safe, wild type mice were administered intraductally with AAV8-RIPI-HNF1A_a or AAV8-RIPII-HNF1A_a vectors. A control group administered intraductally with PBS served as control. Although both vectors promoted specific HNF1A overexpression in islets (FIG. 16), animals treated with AAV8-RIPI-HNF1A_a or AAV8-RIPII-HNF1A_a vectors showed reduced islet number and beta cell mass in comparison with control mice (FIG. 17).

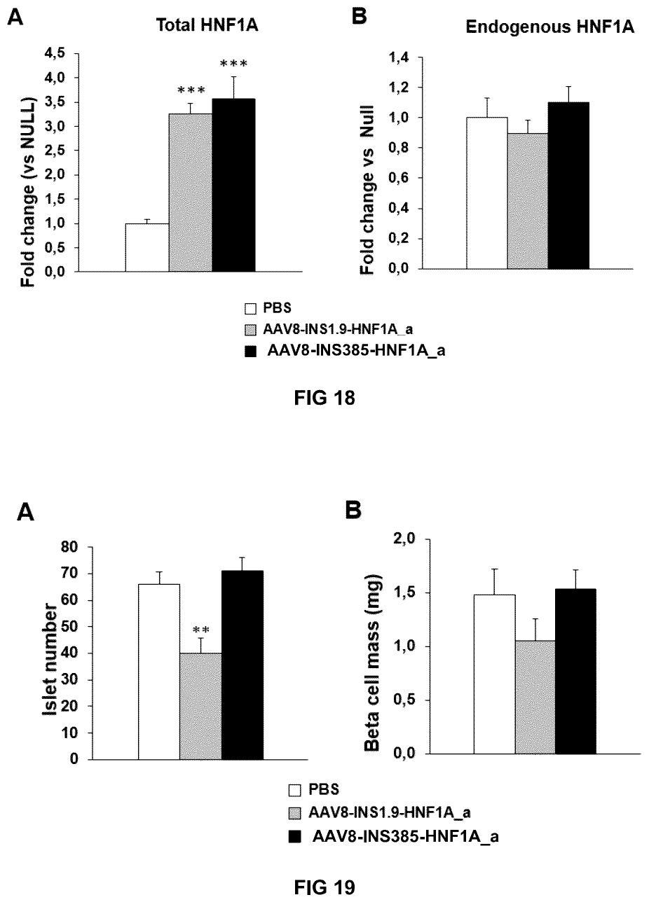

Next, expression cassettes encoding the Mus musculus hepatocyte nuclear factor 1A isoform A (HNF1A_a) under the control of either hINS1.9 or hIns385 promoters and flanked by the inverted terminal repeats (ITRs) of AAV2 were generated (SEQ ID NO: 16 and 17). AAV8 vectors (AAV8-hINS1.9-HNF1A_a and AAV8-hIns385-HNF1A_a) were produced by triple transfection in HEK293 cells. Wild type mice were administered intraductally with AAV8-hINS1.9-HNF1A_α or AAV8-hIns385-HNF1A_a vectors. A control group administered intraductally with PBS served as control. Mice treated intraductally with AAV8-hINS1.9-HNF1A_a or AAV8-hIns385-HNF1A_a vectors showed increased expression levels of HNF1A in islets (FIG. 18). However, mice treated intraductally with AAV8-hINS1.9-HNF1A_a vectors showed decreased number of islets and β-cell mass (FIG. 19). These observations further confirmed the results obtained in WT mice treated intraductally with AAV8-RIPI-HNF1A_a or AAV8-RIPII-HNF1A_a and highlight that high overexpression of HNF1A may cause deleterious effects in β-cells. Therefore, AAV8-hIns385-HNF1A_a were chosen to evaluate the therapeutic efficacy of gene therapy for MODY3.

### Example 8. Reversal of MODY3

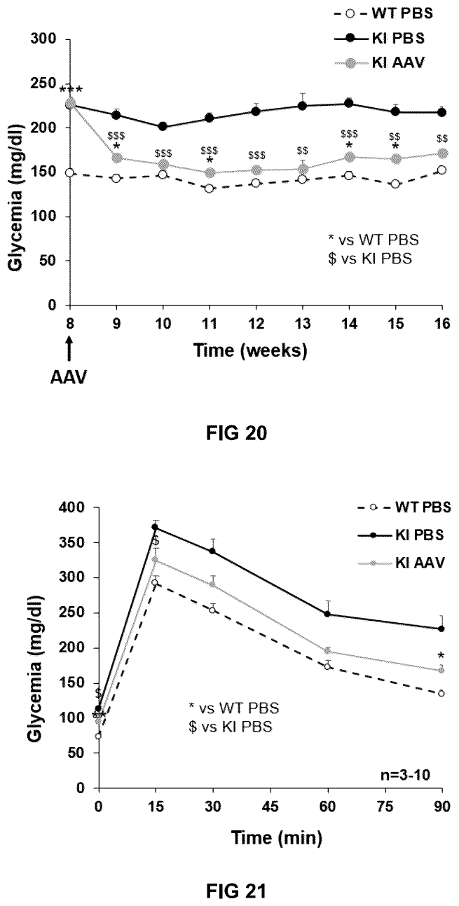

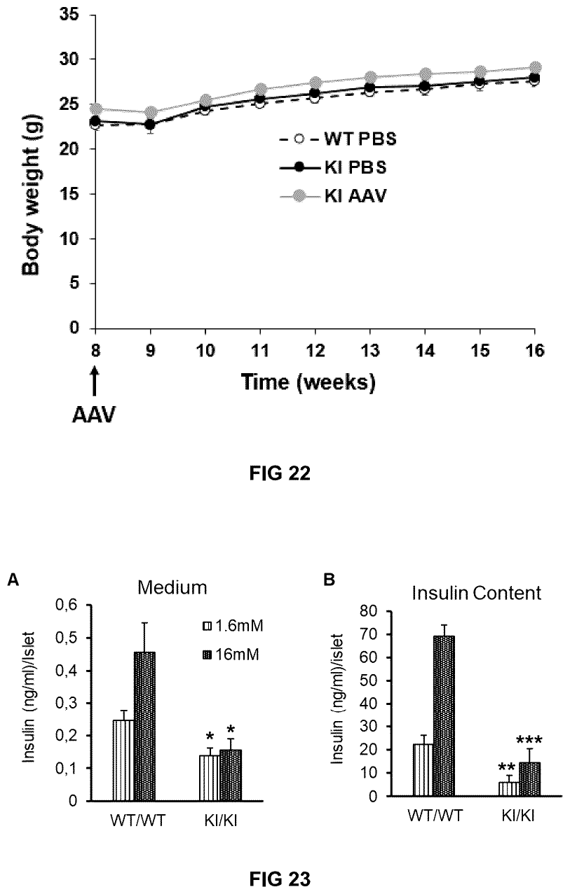

Antidiabetic therapeutic efficacy of AAV8-hIns385-HNF1A_a vectors was evaluated in the MODY3 KI mouse model. Wild type (WT) mice were used as healthy controls, and homozygous KI mice administered with PBS served as MODY3 disease controls. Noticeably, KI MODY3 mice treated with AAV8-hIns385-HNF1A_a vectors showed counteraction of the mild hyperglycemia characteristic of the disease model (FIG. 20). Moreover, MODY3 mice treated with the therapeutic vector also showed improvement of glucose tolerance (FIG. 21). No changes in body weight were observed among experimental groups (FIG. 22).

### Example 9. MODY3 Mice Exhibited Reduced Islet Insulin Content and Impaired Insulin Secretion

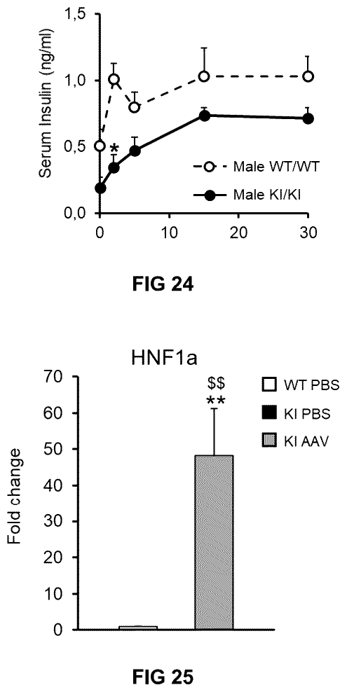

To further phenotype MODY3 KI mice, insulin secretion was evaluated both in vitro and in vivo. To this end, islets from male wild-type and MODY3 mice were incubated with low (1.6 mM) or high glucose (16 mM) and insulin content in islets as well as in the culture medium was analyzed. Islets from MODY3 mice showed decreased insulin content and reduced secretion of insulin into the culture medium at low glucose (FIG. 23). Moreover, while high glucose markedly increased insulin content in WT islets and insulin secretion, this response was blunted in islets from MODY3 mice (FIG. 23). MODY3 mice also showed reduced insulin release in vivo (FIG. 24). In particular, the first phase of insulin secretion in response to glucose was greatly diminished in these mice (FIG. 24), suggesting an impaired secretory response by beta-cells.

### Example 10. Increased HNF1A Expression and Protein Content in Islets from MODY3 Mice Treated with AAV8-hIns385-HNF1A_a Vectors

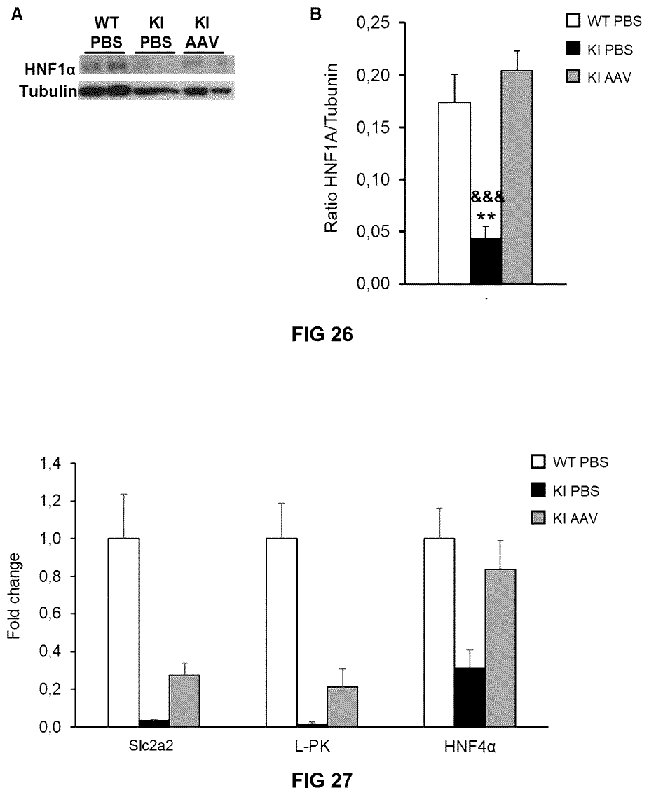

HNF1A expression levels and protein content were analyzed in islet samples from 14 to 16-week-old male wild-type, MODY3 and MODY3 mice treated with AAV8-hINS385-mmHNF1α vectors. MODY3 mice treated with AAV8-hIns385-HNF1A_a vectors showed markedly increased HNF1A expression levels and HNF1A protein content in islets compared with MODY3 mice treated intraductally with PBS (FIGS. 25 and 26). Noticeably, HNF1A protein content in islets was normalized by the AAV treatment (FIG. 26). In addition, expression of the HNF1A gene targets Slc2a2 (encoding for glucose transporter 2, GLUT2), L-pk (L-pyruvate kinase) and Hnf4a (hepatocyte nuclear factor 4 alpha), was also increased in MODY3 mice treated with AAV8-hIns385-HNF1A_a vectors (FIG. 27).

### Example 11. MODY3 Mice Treated with AAV8-hIns385-HNF1A_a Vectors Exhibited Improved Fasted Mild Hyperglycemia

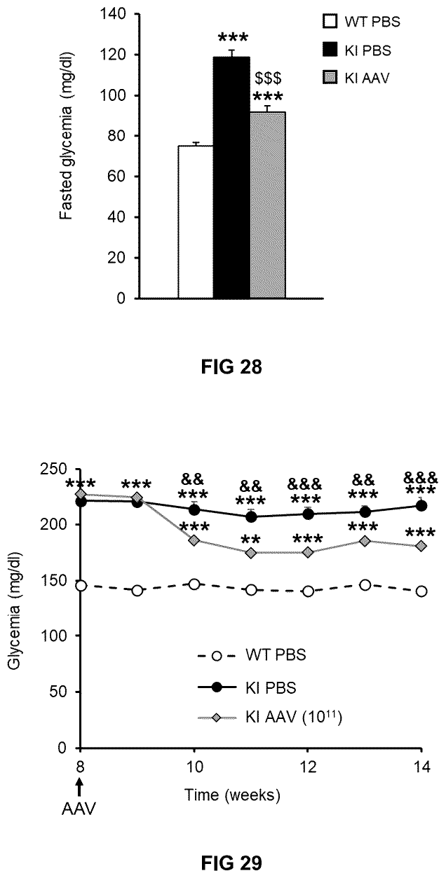

In agreement with counteraction of mild fed hyperglycemia (FIG. 20), male MODY3 mice treated with AAV8-hIns385-HNF1A_a vector also showed markedly reduced glycemia under fasted conditions (FIG. 28).

### Example 12. Reversal of MODY3 at Lower AAV Dose

Next, antidiabetic therapeutic efficacy of AAV8-hIns385-HNF1A_a vectors was evaluated in the MODY3 KI mouse model at a lower dose. Wild type (WT) mice were used as healthy controls, and homozygous KI mice administered with PBS served as MODY3 disease controls. Noticeably, MODY3 KI mice treated with AAV8-hIns385-HNF1A_a vectors at 10¹¹ vg/mouse showed counteraction of the mild hyperglycemia characteristic of the disease model (FIG. 29), similarly to treatment with higher 5×10¹¹ vg/animal dose (FIG. 20).

### Example 13. Downregulation of Hn4a Expression in Islets from MODY1 Mice

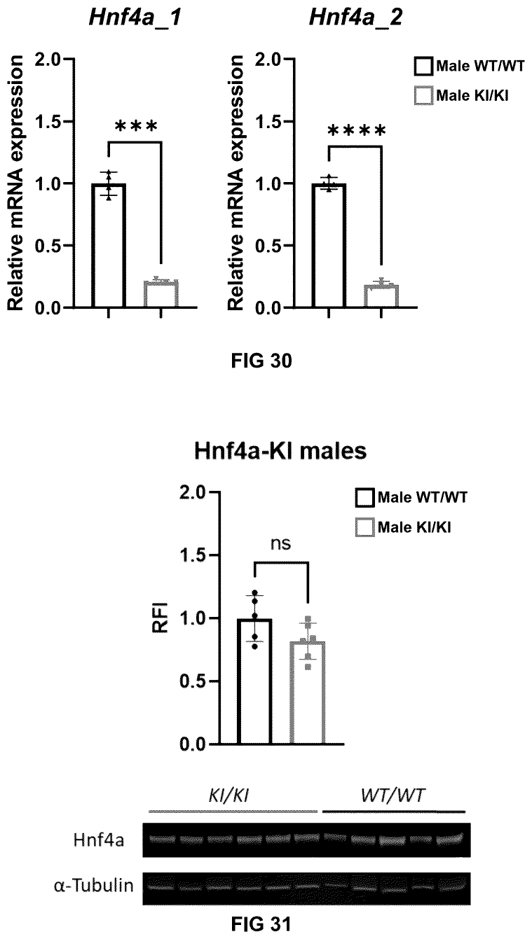

Hnf4a expression levels were analyzed using two different primer pairs in islet samples from 12-14-week-old MODY1 mice. Homozygous MODY1 mice showed significantly reduced Hnf4a expression levels in islets (FIG. 30), whereas HNF4A protein content in the liver of MODY1 mice was not changed (FIG. 31).

### Example 14. Maintenance of Body Weight in MODY1 Mice

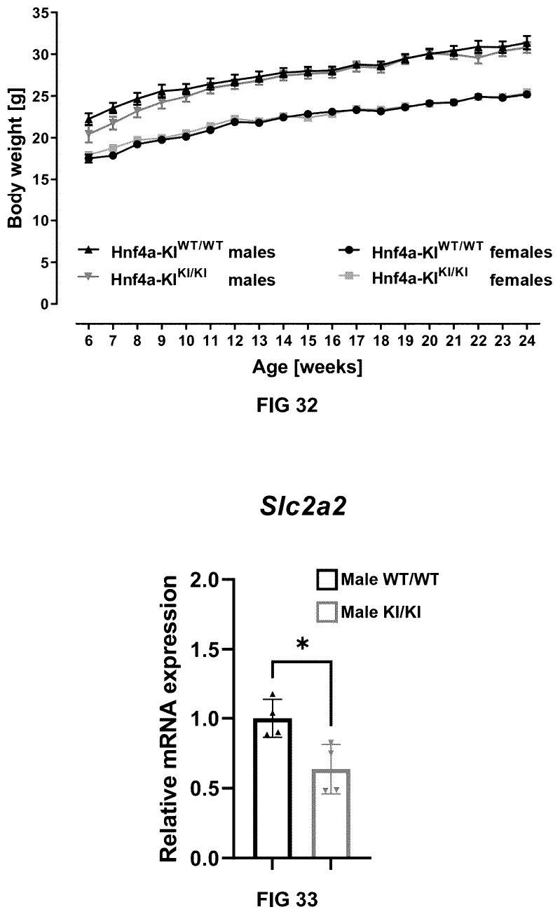

Weekly measurements of the body weight of wild-type (WT/WT) and homozygous MODY1 (KI/KI) mice of either sex show no changes in body weight gain over the period of 6-24 weeks (FIG. 32).

### Example 15. MODY1 Mice Showed Downregulation of HNF4A Target Gene Slc2a2 in Islets

HNF4A was shown to regulate the expression of genes involved β-cell function, among them Slc2a2, which encodes for the glucose transporter 2 (Wang H. et al. (2000), J. Biol. Chem. 275(47), 35953-35959). The expression of Slcs2a2 was significantly downregulated in islets from homozygous MODY1 (KI/KI) compared to islets from wild-type (WT/WT) littermates (FIG. 33).

### Sequences

| SEQ ID NO: | Description of the sequence |
|---|---|
| 1 | Nucleotide sequence of homo sapiens HNF1A |
| 2 | Nucleotide sequence of homo sapiens HNF4A |
| 3 | Nucleotide sequence of mus musculus HNF1A |
| 4 | Nucleotide sequence of mus musculus HNF4A |
| 5 | Nucleotide sequence of canis lupus familiaris HNF1A |
| 6 | Nucleotide sequence of canis lupus familiaris HNF4A |
| 7 | Target sequence of mir-375 |
| 8 | sgRNA for MODY3 model |
| 9 | sgRNA for MODY1 model |
| 10 | rat insulin promoter 1 |
| 11 | rat insulin promoter 2 |
| 12 | full-length human insulin promoter (hINS1.9) |
| 13 | positions −385 to −1 in the human insulin promoter |
| 14 | RIPI-HNF1A gene construct |
| 15 | RIPII- HNF1A gene construct |
| 16 | hIns1.9- HNF1A gene construct |
| 17 | hIns385- HNF1A gene construct |
| 18, 19 | Forward and reverse primers targeting exon 10 and the 3′ UTR of the HNF4A gene |
| 20, 21 | Forward and reverse primers targeting exon 10 and the 3′ UTR of the HNF1A gene |
| 22, 23 | Rplp0 forward and reverse primers |
| 24, 25 | L-PK forward and reverse primers |
| 26, 27 | HNF4a forward and reverse primers |
| 28, 29 | HNF1a forward and reverse primers |
| 30, 31 | Slc2a2 forward and reverse primers |
| 32, 33 | Atp5b forward and reverse primers |
| 34, 35 | Rpl13a forward and reverse primers |
| 36, 37 | Hnf4a_1 forward and reverse primers |
| 38, 39 | Hnf4a_2 forward and reverse primers |
| 40, 41 | Slc2a2 forward and reverse primers |

1. A genetically modified plant or non-human animal having reduced expression of an endogenous target gene, wherein the genome of the plant or non-human animal is modified by ectopic integration of at least one copy of a target sequence of a microRNA.

2. The genetically modified plant or non-human animal according to claim 1, wherein the ectopic integration site is located:
in the 5′ UTR of the target gene or in the region between the 5′ UTR and the first exon of the target gene; or
in the 3′ UTR of the target gene or in the region between the last exon and the 3′ UTR of the target gene.

3. The genetically modified plant or non-human animal according to claim 1, wherein the ectopic integration site is located in the 3′ UTR of the target gene or in the region between the last exon and the 3′ UTR of the target gene.

4. The genetically modified plant or non-human animal according to claim 1, wherein the genome comprises one, two, three, four, five, six, seven, eight or more copies, preferably two, three or four copies, more preferably two copies of the target sequence of a microRNA.

5. The genetically modified plant or non-human animal according to claim 1, wherein the reduced expression is specifically in one or more target cells, tissues and/or organs of an organism, and wherein the target sequence is of a microRNA expressed specifically in said one or more target cells, tissues and/or organs.

6. The genetically modified plant or non-human animal according to claim 1, wherein the plant or non-human animal is a non-human animal, preferably a mammal such as a rodent, more preferably a rat or a mouse, most preferably a mouse.

7. The genetically modified plant or non-human animal according to claim 1, wherein the reduced expression is specifically in the pancreas and wherein the target sequence is of a microRNA expressed specifically in the pancreas, preferably wherein the microRNA is selected from the group consisting of: mir-200a, mir-96, mir-1839-1, mir-34a, mir-7b, mir-7-1, mir-7-2, mir-184, mir-375, mir-219a-1, mir-574, mir-802, mir-152 and mir-148a, more preferably wherein the microRNA is mir-375.

8. The genetically modified plant or non-human animal according to claim 1, wherein the target gene is selected from the group consisting of: HNF4A, GCK, HNF1A, PDX1, HNF1B, NEUROD1, KLF11, CEL, PAX4, INS, BLK, ABCC8, APPL1 and KCNJ11, preferably wherein the target genes is HNF1A or HNF4A.

9. The genetically modified plant or non-human animal according to claim 1, which is a plant or non-human animal disease model, preferably a model for a disease associated with or caused by mutations in the target gene, more preferably wherein the disease is a monogenic disease.

10. The genetically modified plant or non-human animal according to claim 9, wherein the disease is maturity onset diabetes of the young type 3 (MODY3) and the target gene is HNF1A, or wherein the disease is maturity onset diabetes of the young type 1 (MODY1) and the target gene is HNF4A.

11. Method for obtaining a genetically modified plant or non-human animal as described in claim 1, comprising:
(a) providing a cell of said plant or non-human animal;
(b) genetically modifying the cell by ectopic integration of at least one copy of a target sequence of a microRNA in the genome of the cell;
(c) generating an embryo from the cell; and
(d) growing said embryo to form a genetically modified plant or non-human animal.

12. The method according to claim 11, wherein the at least one copy of a target sequence of a microRNA is introduced by homology-directed repair (HDR), preferably CRISPR-mediated

13. The method according to claim 11, further comprising the step of back-crossing the genetically modified plant or non-human animal with non-genetically modified wildtype plant or non-human animal.

14. Method for identifying and/or evaluating therapeutic efficacy of a candidate agent for treating, inhibiting or preventing a disease, comprising administering the candidate agent to a plant or non-human animal as described in claim 8, or a cell, tissue or organ derived thereof.

15. (canceled)


SEQUENCE LISTING (41 sequences)

SEQ ID NO: 1 - DNA, 1896 bp, Homo sapiens
atggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacgggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccagcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagtaa

SEQ ID NO: 2 - DNA, 1425 bp, Homo sapiens
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 3 - DNA, 1887 bp, Mus musculus
atggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaa

SEQ ID NO: 4 - DNA, 1425 bp, Mus musculus
atgcgactctctaaaacccttgccggcatggatatggccgactacagcgctgccctggacccagcctacaccaccctggagtttgaaaatgtgcaggtgttgaccatgggcaatgacacgtccccatctgaaggtgccaacctcaattcatccaacagcctgggcgtcagtgccctgtgcgccatctgtggcgaccgggccaccggcaaacactacggagcctcgagctgtgacggctgcaaggggttcttcaggaggagcgtgaggaagaaccacatgtactcctgcaggtttagccgacaatgtgtggtagacaaagataagaggaaccagtgtcgttactgcaggcttaagaagtgcttccgggctggcatgaagaaggaagctgtccaaaatgagcgggaccggatcagcacgcggaggtcaagctacgaggacagcagcctgccctccatcaacgcgctcctgcaggcagaggttctgtcccagcagatcacctctcccatctctgggatcaatggcgacattcgggcaaagaagattgccaacatcacagacgtgtgtgagtctatgaaggagcagctgctggtcctggtcgagtgggccaagtacatcccggccttctgcgaactccttctggatgaccaggtggcgctgctcagggcccacgccggtgagcatctgctgcttggagccaccaagaggtccatggtgtttaaggacgtgctgctcctaggcaatgactacatcgtccctcggcactgtccagagctagcggagatgagccgtgtgtccatccgcatcctcgatgagctggtcctgcccttccaagagctgcagattgatgacaatgaatatgcctgcctcaaagccatcatcttctttgatccagatgccaaggggctgagtgacccgggcaagatcaagcggctgcggtcacaggtgcaagtgagcctggaggattacatcaacgaccggcagtacgactctcggggccgctttggagagctgctgctgctgttgcccacgctgcagagcatcacctggcagatgatcgaacagatccagttcatcaagctcttcggcatggccaagattgacaacctgctgcaggagatgcttctcggagggtctgccagtgatgcaccccacacccaccaccccctgcaccctcacctgatgcaagaacacatgggcaccaatgtcattgttgctaacacgatgccctctcacctcagcaatggacagatgtgtgagtggccccgacccagggggcaggcagccactcccgagactccacagccatcaccaccaagtggctcgggatctgaatcctacaagctcctgccaggagccatcaccaccatcgtcaagcctccctctgccattccccagccaacgatcaccaagcaagaagccatctag

SEQ ID NO: 5 - DNA, 1896 bp, Canis lupus familiaris
atggtttctaaactgagccagctgcagacggagctcctggcggccctgctccactcgggcctgagcaaagaggcactgatccaggcgttgggtgagccggggccctacctgctggctggagatggccccctggacaagggggagtcctgcggcggcggtcggggggagctggccgagctgcccaacgggctgggggaggcgaggggctccgaggacgagacggacgacgatggggaagacttcacaccgcccatcctcaaggagctggagaacctcagccccgaggaggcggcccaccagaaagccgtggtggagaccttgctgcaggaggacccgtggcgcgtggccaagatggtcaaatcctacctgcagcagcataacatcccacagcgggaggtagtcgacaccactggcctcaaccagtcacacttgtcccagcatctcaacaagggcacccccatgaagacacagaagcgagctgccctgtacacctggtacgtccgcaagcagcgagaggtggcccagcagttcacccacgcagggcaaggtgggctgattgaagagcccacaggtgatgagctgccaaccaagaaggggcggaggaatcgtttcaagtggggcccagcctcccagcagatcctgttccaggcctatgagaggcagaagaaccccagcaaggaggagcgagaggcattggtggaggagtgcaatagggcggaatgcatccagaggggggtgtcaccatcgcaggcacaggggctgggctccaaccttgtcacagaggtgcgtgtctacaactggtttgccaatcgccgcaaggaagaagcctttcgtcataagctggccatggacacatacagcgggccaccacctgggccaggcccgggccccacactgcctgcccacagctctccgggcttgcccccacctgccctctcccctagtaaggtccacggtgtgcgctatggacagcaagcaactagcgagggggcggaagtgccttcgagcagcggtggtcccttggtgacggtgtctgcgcccctgcaccaagtgtcccccacaggcctggagcccagtcacagtctgctgagcaccgaagccaagctggtctcagcaactgggggccccttgccccctgtcagcaccctgacagcactgcacagcttggagcagacctccccaggcctcaaccaacagccccagaatctcatcatggcctcacttcccggggtcatggccattgggcctggtgagcctgcctccctgggccccacgttcaccaacacgggtgcctccaccctggtcattggcctggcctccactcaggcccagagcgtgccagtcatcaacagcatgggcagcagcctgaccaccctgcagccagtccagttctcccagccactgcacccttcctaccagcagccactcatgtctcccatgcagagccatgtggcccagagccccttcatggccaccatggcccagattcagagcccccatgccctctacagccacaagcccgaggtggcccagtacacccacacgggcctgcttccgcagaccatgctcatcaccgacactaccaacctgagcgccctggccagcctcacgcccaccaagcaggtcttcacctcagatgctgaggcctccagtgagtccgggcttcatacgccggcgtctcagaccaccaccatccacatccccagccaggatcctgttggcatccagcacctgcagcctgcccaccggctcagcgccagccccaccgtgtcctccagcagcctggtgttgtaccagagctccgactccaacaacggccacagccacctgttgccatccaaccacagcgtcatcgagaccttcatttccacgcagatggcttcatcctcccagtaa

SEQ ID NO: 6 - DNA, 1455 bp, Canis lupus familiaris
atgcgcctctccaaaaccctcgtcgacatggacatggctgactacagtgccgcgctggacccagcctacaccacccttgagttcgagaacgtgcaggtgttgacgatgggcaatgacacatccccatccgaaggtgccaacctcaacgcacccaacagcctgggggtcagcgctctgtgtgccatctgtggggaccgggccacagggaaacactacggtgcctcgagctgtgacggttgcaagggcttcttccggaggagcgtgagaaagaaccacatgtactcctgcaggtttagtcggcaatgcgtcgtggacaaagacaagaggaaccagtgtcgttactgcaggctaaagaagtgcttccgggctggcatgaagaaggaagctgtgcaaaatgagcgggaccggatcagcactcgccggtcaagctatgaggacagcagcctgccctccatcaatgcattgctgcaggccgaggtcctgtcccagcagatcacctcccctgtctctgggatcaacggagacattcgggcgaagaagatcgccagcatcgcagacgtgtgtgagtccatgaaggagcagctgctggtgctcgttgagtgggccaagtacattcctgctttctgtgagctcccgctggatgaccaggtggccctgctcagagcccatgccggtgagcacctgctacttggagccaccaagcgatccatggtgttcaaggatgttctgcttctaggcaatgactacatcgtccctcgccactgcccagagctggccgaaatgagccgggtgtccatgcgcatcctcgacgagttggtgctgcccttccaggagctacagatcgatgacaatgagtatgcctgcctcaaagccatcatcttctttgacccagatgccaaggggctgagcgacccgggcaagatcaagcggctgcgctcgcaggtgcaggtgagcctggaggactacatcaacgaccgccagtatgactcccgaggccgcttcggtgagctgctgctcctgctgcccaccctgcaaagcatcacctggcagatgatcgagcagatccagttcatcaagctcttcggcatggccaagattgacaacctgctgcaggagatgttgctgggaggtccgtgccaggcccaggaggggctgagagggtcctccagtgatgcacctcatgcccatcatcccctgcatcctcatctgatgcaggaacacatgggcacaaatgtcattgttgccaacacgatgccctctcacctcagcaatggacagatgtgtgagtggccccgacccagggggcaggcagccacccctgagactccacagccctcgccgccaggtggctcagggtctgagccctacaagctcctgccaggagccattgccacaatcgtcaagcccccctccgccatcccccagccgaccatcaccaagcaggaagtcatctag

SEQ ID NO: 7 - DNA, 22 bp, Artificial sequence
  Target sequence of mir-375
tctcgcgagccgaacgaacaaa

SEQ ID NO: 8 - RNA, 20 nt, Artificial sequence
  sgRNA for MODY3 model
ccuccucuucccaguaaccg

SEQ ID NO: 9 - RNA, 20 nt, Artificial sequence
  sgRNA for MODY1 model
aagccaucuagcaagcugcu

SEQ ID NO: 10 - DNA, 539 bp, Artificial sequence
  rat insulin promoter 1
cgccgggttttgtggaagtagagatagaggagaagggaccattacatgtcctgctgcctgagttctgctttccttctccctttgaaggtgagctggggtctcagctgagctaagaatccagctatcaatagaaactatgaaacagttccagggacaaagataccaggtccccaacaactgcaactttctgggaaatgaggtggaaagtgctcagccaaggaaaaagagggccttaccctctctgggacaatgattgtgctgtgaactgcttcatcaggccatctggccccttgttaataatctaattaccctaggtctaagtagagttgctgacgtccaatgagcgctttctgcagacttagcactaggcaagtgtttggaaattacggcttcggcccctctcgccatctgcctacctacccctcctagagcccttaatgggccaaacggcaaagtccagggggcagagaggaggtgctttggactataaagctagtggagacccagtaactcccaaccctaacgggatatcctcgagt

SEQ ID NO: 11 - DNA, 721 bp, Artificial sequence
  rat insulin promoter 2
gatcccccaaccactccaagtggaggctgagaaaggttttgtagctgggtagagtatgtactaagagatggagacagctggctctgagctctgaagcaagcacctcttatggagagttgctgaccttcaggtgcaaatctaagatactacaggagaatacaccatggggcttcagcccagttgactcccgagtgggctatgggtttgtggaaggagagatagaagagaagggacctttcttcttgaattctgctttccttctacctctgagggtgagctggggtctcagctgaggtgaggacacagctatcagtgggaactgtgaaacaacagttcaagggacaaagttactaggtcccccaacaactgcagcctcctggggaatgatgtggaaaaatgctcagccaaggacaaagaaggcctcaccctctctgagacaatgtcccctgctgtgaactggttcatcaggccacccaggagcccctattaagactctaattaccctaaggctaagtagaggtgttgttgtccaatgagcactttctgcagacctagcaccaggcaagtgtttggaaactgcagcttcagcccctctggccatctgctgatccacccttaatgggacaaacagcaaagtccaggggtcagggggggggtgctttggactataaagctagtggggattcagtaacccccagccctaagatatcctcgagtctag

SEQ ID NO: 12 - DNA, 1900 bp, Artificial sequence
  full-lenght human insulin promoter (hINS1.9)
ccccggccgacaacactggcaaactcctactcatccacgaaggccctcctgggcatggtggtccttcccagcctggcagtctgttcctcacacaccttgttagtgcccagcccctgaggttgcagctgggggtgtctctgaagggctgtgagcccccaggaagccctggggaagtgcctgccttgcctccccccggccctgccagcgcctggctctgccctcctacctgggctccccccatccagcctccctccctacacactcctctcaaggaggcacccatgtcctctccagctgccgggcctcagagcactgtggcgtcctggggcagccaccgcatgtcctgctgtggcatggctcagggtggaaagggcggaagggaggggtcctgcagatagctggtgcccactaccaaacccgctcggggcaggagagccaaaggctgggtgtgtgcagagcggccccgagaggttccgaggctgaggccagggtgggacatagggatgcgaggggccggggcacaggatactccaacctgcctgcccccatggtctcatcctcctgcttctgggacctcctgatcctgcccctggtgctaagaggcaggtaggggctgcaggcagcagggctcggagcccatgccccctcaccatgggtcaggctggacctccaggtgcctgttctggggagctgggagggccggaggggtgtaccccaggggctcagcccagatgacactatgggggtgatggtgtcatgggacctggccaggagaggggagatgggctcccagaagaggagtgggggctgagagggtgcctggggggccaggacggagctgggccagtgcacagcttcccacacctgcccacccccagagtcctgccgccacccccagatcacacggaagatgaggtccgagtggcctgctgaggacttgctgcttgtccccaggtccccaggtcatgccctccttctgccaccctggggagctgagggcctcagctggggctgctgtcctaaggcagggtgggaactaggcagccagcagggaggggacccctccctcactcccactctcccacccccaccaccttggcccatccatggcggcatcttgggccatccgggactggggacaggggtcctggggacaggggtgtggggacaggggtcctggggacaggggtctggggacaggggtcctggggacaggggtgtggggacaggggtgtggggacaggggtgtggggacaggggtcctggggacaggggtctggggacaggggtctgaggacaggggtgtggggacaggggtgtggggacaggggtgtggggacaggggtgtggggacaggggtctggggacaggggtccgggggacaggggtgtggggacaggggtgtggggacaggggtgtggggacaggggtctggggacaggggtgtggggacaggggtcctggggacaggggtgtggggataggggtgtggggacaggggtgtggggacaggggtgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctc

SEQ ID NO: 13 - DNA, 555 bp, Artificial sequence
  positions -385 to -1 in the human insulin promoter
tgtggggacaggggtctggggacaggggtccgggggacaggggtgtggggacaggggtgtggggacaggggtgtggggacaggggtctggggacaggggtgtggggacaggggtcctggggacaggggtgtggggataggggtgtggggacaggggtgtggggacaggggtgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctc

SEQ ID NO: 14 - DNA, 2730 bp, Artificial sequence
  RIPI-HNF1A gene construct
cgccgggttttgtggaagtagagatagaggagaagggaccattacatgtcctgctgcctgagttctgctttccttctccctttgaaggtgagctggggtctcagctgagctaagaatccagctatcaatagaaactatgaaacagttccagggacaaagataccaggtccccaacaactgcaactttctgggaaatgaggtggaaagtgctcagccaaggaaaaagagggccttaccctctctgggacaatgattgtgctgtgaactgcttcatcaggccatctggccccttgttaataatctaattaccctaggtctaagtagagttgctgacgtccaatgagcgctttctgcagacttagcactaggcaagtgtttggaaattacggcttcggcccctctcgccatctgcctacctacccctcctagagcccttaatgggccaaacggcaaagtccagggggcagagaggaggtgctttggactataaagctagtggagacccagtaactcccaaccctaacgggatgatatcctcgaggctagcgaattcgccaccatggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaaggtaccgcggccgcgttaacacgcgtgatatccacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 15 - DNA, 2906 bp, Artificial sequence
  RIPII- HNF1A gene construct
ggatcccccaaccactccaagtggaggctgagaaaggttttgtagctgggtagagtatgtactaagagatggagacagctggctctgagctctgaagcaagcacctcttatggagagttgctgaccttcaggtgcaaatctaagatactacaggagaatacaccatggggcttcagcccagttgactcccgagtgggctatgggtttgtggaaggagagatagaagagaagggacctttcttcttgaattctgctttccttctacctctgagggtgagctggggtctcagctgaggtgaggacacagctatcagtgggaactgtgaaacaacagttcaagggacaaagttactaggtcccccaacaactgcagcctcctggggaatgatgtggaaaaatgctcagccaaggacaaagaaggcctcaccctctctgagacaatgtcccctgctgtgaactggttcatcaggccacccaggagcccctattaagactctaattaccctaaggctaagtagaggtgttgttgtccaatgagcactttctgcagacctagcaccaggcaagtgtttggaaactgcagcttcagcccctctggccatctgctgatccacccttaatgggacaaacagcaaagtccaggggtcagggggggggtgctttggactataaagctagtggggattcagtaacccccagccctaagatatcctcgaggctagcgaattcgccaccatggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaaggtaccgcggccgcgttaacacgcgtgatatccacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 16 - DNA, 4101 bp, Artificial sequence
  hIns1.9- HNF1A gene construct
ccccggccgacaacactggcaaactcctactcatccacgaaggccctcctgggcatggtggtccttcccagcctggcagtctgttcctcacacaccttgttagtgcccagcccctgaggttgcagctgggggtgtctctgaagggctgtgagcccccaggaagccctggggaagtgcctgccttgcctccccccggccctgccagcgcctggctctgccctcctacctgggctccccccatccagcctccctccctacacactcctctcaaggaggcacccatgtcctctccagctgccgggcctcagagcactgtggcgtcctggggcagccaccgcatgtcctgctgtggcatggctcagggtggaaagggcggaagggaggggtcctgcagatagctggtgcccactaccaaacccgctcggggcaggagagccaaaggctgggtgtgtgcagagcggccccgagaggttccgaggctgaggccagggtgggacatagggatgcgaggggccggggcacaggatactccaacctgcctgcccccatggtctcatcctcctgcttctgggacctcctgatcctgcccctggtgctaagaggcaggtaggggctgcaggcagcagggctcggagcccatgccccctcaccatgggtcaggctggacctccaggtgcctgttctggggagctgggagggccggaggggtgtaccccaggggctcagcccagatgacactatgggggtgatggtgtcatgggacctggccaggagaggggagatgggctcccagaagaggagtgggggctgagagggtgcctggggggccaggacggagctgggccagtgcacagcttcccacacctgcccacccccagagtcctgccgccacccccagatcacacggaagatgaggtccgagtggcctgctgaggacttgctgcttgtccccaggtccccaggtcatgccctccttctgccaccctggggagctgagggcctcagctggggctgctgtcctaaggcagggtgggaactaggcagccagcagggaggggacccctccctcactcccactctcccacccccaccaccttggcccatccatggcggcatcttgggccatccgggactggggacaggggtcctggggacaggggtgtggggacaggggtcctggggacaggggtctggggacaggggtcctggggacaggggtgtggggacaggggtgtggggacaggggtgtggggacaggggtcctggggacaggggtctggggacaggggtctgaggacaggggtgtggggacaggggtgtggggacaggggtgtggggacaggggtgtggggacaggggtctggggacaggggtccgggggacaggggtgtggggacaggggtgtggggacaggggtgtggggacaggggtctggggacaggggtgtggggacaggggtcctggggacaggggtgtggggataggggtgtggggacaggggtgtggggacaggggtgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcgatatcctcgaggctagcgaattcgccaccatggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaaggtaccgcggccgcgttaacacgcgtgatatccacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 17 - DNA, 2586 bp, Artificial sequence
  hIns385- HNF1A gene construct
tgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcgatatcctcgaggctagcgaattcgccaccatggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaaggtaccgcggccgcgttaacacgcgtgatatccacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 18 - DNA, 20 bp, Artificial sequence
  Forward primer
tagaagagctttccctgggc

SEQ ID NO: 19 - DNA, 20 bp, Artificial sequence
  Reverse primer
ggggtgaagaagttgaggga

SEQ ID NO: 20 - DNA, 20 bp, Artificial sequence
  Forward primer
ggacttggccaacagctagt

SEQ ID NO: 21 - DNA, 20 bp, Artificial sequence
  Reverse primer
ggaggagcagcagtgtcaat

SEQ ID NO: 22 - DNA, 20 bp, Artificial sequence
  Rplp0 forward primer
tcccaccttgtctccagtct

SEQ ID NO: 23 - DNA, 21 bp, Artificial sequence
  Rplp0 reverse primer
actggtctaggacccgagaag

SEQ ID NO: 24 - DNA, 20 bp, Artificial sequence
  L-PK forward primer
gtttcttgggcaacaggaag

SEQ ID NO: 25 - DNA, 20 bp, Artificial sequence
  L-PK reverse primer
aggaggcaaagatgatgtcc

SEQ ID NO: 26 - DNA, 20 bp, Artificial sequence
  HNF4a forward primer
agattgacaacctgctgcag

SEQ ID NO: 27 - DNA, 20 bp, Artificial sequence
  HNF4a reverse primer
tgcccatgtgttcttgcatc

SEQ ID NO: 28 - DNA, 20 bp, Artificial sequence
  HNF1a forward primer
tgtcacagcacctcaacaag

SEQ ID NO: 29 - DNA, 20 bp, Artificial sequence
  HNF1a reverse primer
tgtgggctcttcaatcagtc

SEQ ID NO: 30 - DNA, 20 bp, Artificial sequence
  Slc2a2 forward primer
atcccttggttcatggttgc

SEQ ID NO: 31 - DNA, 20 bp, Artificial sequence
  Slc2a2 reverse primer
aattgcagacccagttgctg

SEQ ID NO: 32 - DNA, 21 bp, Artificial sequence
  Atp5b forward primer
ggtttgaccgttgctgaatac

SEQ ID NO: 33 - DNA, 20 bp, Artificial sequence
  Atp5b reverse primer
taaggcagacacctctgagc

SEQ ID NO: 34 - DNA, 22 bp, Artificial sequence
  Rpl13a forward primer
tgaagcctaccagaaagtttgc

SEQ ID NO: 35 - DNA, 20 bp, Artificial sequence
  Rpl13a reverse primer
gcctgtttccgtaacctcaa

SEQ ID NO: 36 - DNA, 21 bp, Artificial sequence
  Hnf4a_1 forward primer
gttctgtcccagcagatcacc

SEQ ID NO: 37 - DNA, 20 bp, Artificial sequence
  Hnf4a_1 reverse primer
cttctttgcccgaatgtcgc

SEQ ID NO: 38 - DNA, 20 bp, Artificial sequence
  Hnf4a_2 forward primer
cgtgctgctcctaggcaatg

SEQ ID NO: 39 - DNA, 20 bp, Artificial sequence
  Hnf4a_2 reverse primer
catcgaggatgcggatggac

SEQ ID NO: 40 - DNA, 20 bp, Artificial sequence
  Slc2a2 forward primer
ggggacaaacttggaaggat

SEQ ID NO: 41 - DNA, 20 bp, Artificial sequence
  Slc2a2 reverse primer
tgaggccagcaatctgacta